(19) United States
(12) Patent Application Publication
(10) Pub. No.: US 2015/0261923 A1
(43) Pub. Date: Sep. 17, 2015

(54) DIGITAL MEDICAL INTERN SYSTEM

(71) Applicant: Wisconsin Alumni Research Foundation, Madison, WI (US)

(72) Inventor: Joshua Medow, Verona, WI (US)

(21) Appl. No.: 14/206,983

(22) Filed: Mar. 12, 2014

(51) Int. Cl. G06F 19/00; A61B 5/02; A61B 5/0205; A61B 5/00; A61B 5/145

(52) U.S. Cl. CPC G06F 19/345; A61B 5/4875; A61B 5/14532; A61B 5/0205; A61B 5/02028; A61B 5/486; A61B 5/002; A61B 5/7475; A61B 5/021

(57) ABSTRACT

Media, methods, and systems provide a platform for monitoring patient health and recommending treatment in a protocolized manner. The protocol is customizable to fit patient and physician needs and the platform is capable of alerting medical staff when treatment is needed, precluding traditional practices of intensive-monitoring.

### BACKGROUND

The medical care for some critical patients, like those stabilized in preparation for organ donation, requires a continuous and complicated medical decision-making process by caregivers. For example, critically-injured or dying patients are prone to crash often and the steps for stabilizing a patient after a crash must be performed immediately to be effective. For this reason, care of such patients is often overseen directly by a medical intern and/or nursing staff with instructions to contact the supervising physician for situations where the protocol is not clearly defined.

### SUMMARY OF THE DISCLOSURE

One issue with the current system that was recognized by the present inventors is that it relies heavily on the knowledge and attention of the attending staff. Since the staff can only respond to changing medical conditions one patient at a time and in accordance with their personal understanding of the complex protocols, some problems may be overlooked or discovered late into the time for response. This is especially true in the situation where a supervising physician must be contacted before care may proceed. Another issue that the inventors noticed with regard to the current system is that such intense supervision is costly and inefficient for the hospital. Like the previously mentioned issue, the effect of this problem is particularly costly when a supervising physician is required to respond to many situations personally. Since the team caring for patient may change over time, the physician may need to repeat the same instructions multiple times to respond to different team members' concerns.

The present disclosure relates to methods, systems, and media that help to alleviate the problems discussed above. In particular, the presently disclosed embodiments allow for patient management protocols and monitoring to be automated in a customizable format. Such embodiments create a more effective and efficient monitoring system even with limited staff or experience. Present embodiments also allow a supervising physician or other attending staff to preset rules and procedures related to one or multiple patients, avoiding the need for physician intervention in predefined instances.

In one embodiment, an example computer readable medium stores instructions that a processing system may execute in order to perform certain functions. The functions include receiving health data representing patient vital statistics and using preset treatment protocols to recommend a treatment based on the received health data. The treatment protocols identify a medical caregiver associated with the recommended treatment and the functions include automatically causing a notification system to contact the identified medical caregiver in response to recommending the treatment.

In another embodiment, an example method involves receiving health data representing patient vital statistics and using preset treatment protocols to recommend a treatment based on the received health data. The treatment protocols identify a medical caregiver associated with the recommended treatment and the functions include automatically causing a notification system to contact the identified medical caregiver in response to recommending the treatment.

In another embodiment, an example processing system includes a processor, a computer readable medium, and a communication interface. The processor is configured to receive health data representing patient vital statistics and, using treatment protocols stored in the computer-readable medium, recommend a treatment based on the received health data. The processor is also configured to automatically causing a notification system to contact the identified medical caregiver via the communication interface, in response to recommending a treatment that indicates the medical caregiver.

### BRIEF DESCRIPTION OF THE DRAWINGS

FIG. 1 is a schematic illustrating elements of an example network, according to an example embodiment.

FIG. 2 is a schematic illustrating elements of an example processing system according to an example embodiment.

FIG. 3 is a block diagram illustrating a method according to an example embodiment.

FIG. 4 is a block diagram illustrating another method according to an example embodiment.

FIGS. 5A and 5B are a block diagram illustrating an example set of medical protocols for illustration.

FIGS. 6A-6D are a block diagram illustrating an example set of medical protocols.

FIGS. 7A and 7B are a block diagram illustrating an example set of medical protocols.

FIG. 8 is a block diagram illustrating an example set of medical protocols for an adjustment procedure.

FIGS. 9A-9D are a block diagram illustrating an example set of medical protocols.

FIGS. 10-18 are block diagrams illustrating a main algorithm routine (FIG. 10) and various subroutines (FIGS. 11A-18) of an example medical procedure.

### DETAILED DESCRIPTION

### I. Example System Architecture

Functions and procedures described herein may be executed according to any of several embodiments. For example, procedures may be performed by specialized equipment that is designed to perform the particular functions. As another example, the functions may be performed by general-use equipment that executes commands related to the procedures. As still another example, each function may be performed by a different piece of equipment with one piece of equipment serving as control or with a separate control device. As a further example, procedures may be specified as program instructions on a computer-readable medium.

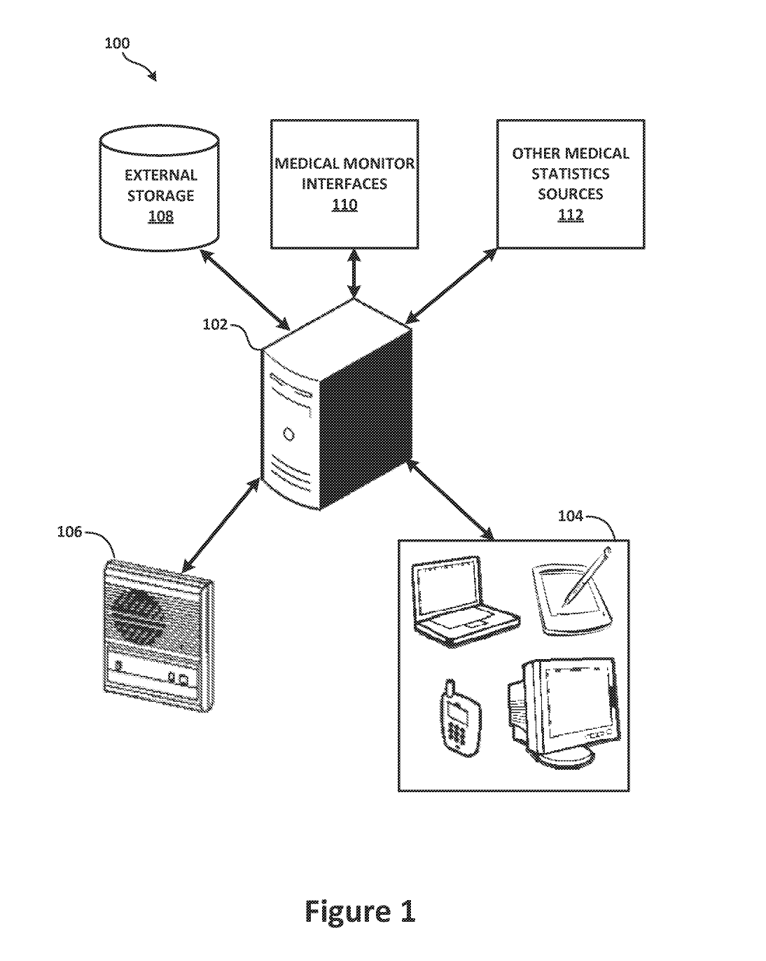

FIG. 1 shows a networked system 100 according to an exemplary embodiment. As shown, the system includes a processing system 102 communicatively coupled to a set of remote devices. In some embodiments, like the network shown in FIG. 1, processing system 102 may connect to various output systems, such as networked devices 104 and intercom 106. Processing system 102 may also connect with various information input devices through the external storage 108, medical monitor interfaces 110, and/or other medical statistics sources 112. Communicative links are formed between each of the elements of system 100. Such links may be any type of communicative connection. For example, the connections may be wired electrical connections, fiber-optic connections, air interfaces, or acoustic transmission networks.

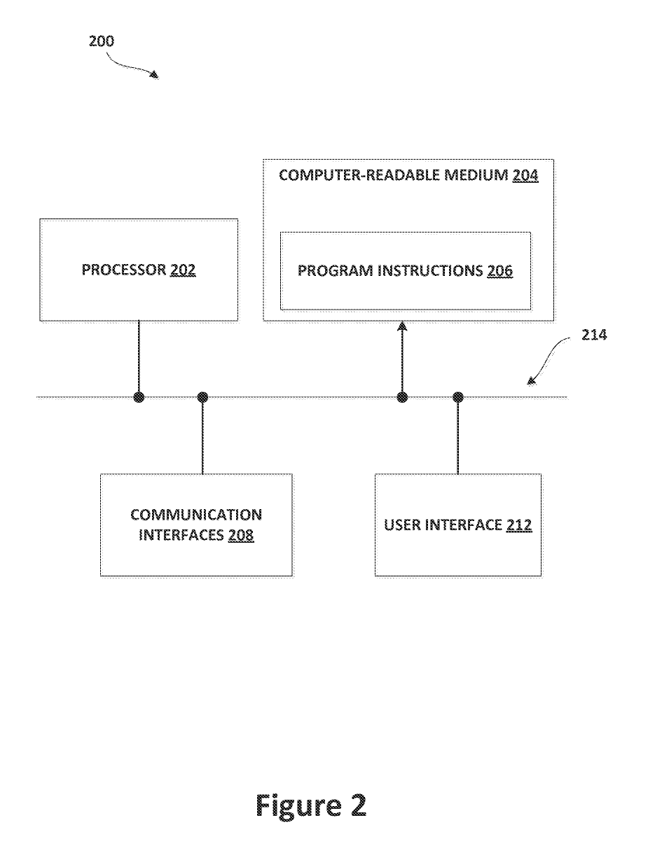

Processing system 102 may be any generalized computing device that stores instructions for carrying out an exemplary process. Alternatively, processing system 102 may be a specialized computing device configured to perform the certain functions needed with hardware. In still other embodiments, processing system 102 may be a set of various computing devices, either performing the same function or each configured to perform a specific function. Processing system 102 may typically include a computer-readable medium, processor, and communication interfaces among other example components, as described with reference to FIG. 2.

As shown in FIG. 1, remote devices 104 may be any of various device types. Devices 104 may be associated with one or more network locations on any of various communication networks. As will be explained in more detail below, external devices 104 may be contacted through a medical notification system in order to alert or contact a caregiver. For example, if an external device 104 is a cellular phone, then the network address may be a telephone number and the notification system may access a landline or cellular telephone network to contact external device 104. As another example, if the devices an Internet-enabled computing device, then the network location may be a registration node on an email, VoIP, or other registration server with which the notification system may communicate over various data links. Any presently known or future device capable of these functions may be used as a device 104. Some non-exclusive examples include, e-readers, tablets, laptops, smartphones, video phones, televisions, desktop computers, PDAs, pagers, and/or fax machines. In some system 106 may be any of various internal communication systems. For example, intercom 106 may include speakers throughout a hospital or campus along with supporting audio and control electronics. Some cases, playback devices, or text-to-speech processors may connect directly into the intercom system, allowing processing system 102 to initiate automated intercom messages without requiring human interaction. In other cases, a switchboard may receive notification requests and present the notification to a user for reading over the intercom system.

As will be explained more below, processing system 102 may receive health data and/or protocols from various sources. Although FIG. 1 shows three main types of information sources, this illustration is not meant to be limiting. Health data and protocol information may be received in ways other than those explicitly shown and described. As shown in FIG. 1, processing system 102 may receive some data from, and transfer data to, external storage 108. External storage 108 may store previously determined health information for continually updated information from sources not directly connected to processing system 102. Also shown in FIG. 1, data sources may include medical monitor interfaces for directly or indirectly connecting processing system 102 may connect to one or more medical diagnostic tools and equipment. For example, medical monitors may include heart monitors, but pressure and characteristics mongers, respiration mongers, brain activity monitors, and muscle activity monitors, among other examples. Also as shown in FIG. 1, other medical statistics sources 112 may connect with processing system 102. For example, medical statistics sources may include other monitoring programs, medical diagnostic software, user interfaces, scanning devices, sources, and general communication networks, and/or other types of interfaces.

One example processing system (200) is shown in FIG. 2. As shown, processing system 200 includes processor 202, computer-readable medium (CRM) 204, communication interfaces 208, and user interface 212 all connected through system bus 214. Also as shown, program instructions 206 are stored on computer-readable medium 204. In the present disclosure, this device may be seen as an embodiment of processing system 102.

Processor 202 may include any processor type capable of executing program instructions 206 in order to perform the functions described herein. For example, processor 202 may be any general-purpose processor, specialized processing unit, or device containing processing elements. In some cases, multiple processing units may be connected and utilized in combination to perform the various functions of processor 202.

CRM 204 may be any available media that can be accessed by processor 202 and any other processing elements in device 200. By way of example, CRM 204 may include RAM, ROM, EPROM, EEPROM, CD-ROM or other optical disk storage, magnetic disk storage or other magnetic storage devices, or any other medium that can be used to carry or store desired program code in the form of program instructions or data structures, and which can be executed by a processor. When information is transferred or provided over a network or another communications connection (either hardwired, wireless, or a combination of hardwired or wireless) to a machine, the machine properly views the connection as a CRM. Thus, any such connection to a computing device or processor is properly termed a CRM. Combinations of the above are also included within the scope of computer-readable media.

Program instructions 206 may include, for example, instructions and data capable of causing a processing unit, a general-purpose computer, a special-purpose computer, special-purpose processing machines, or remote server systems to perform a certain function or group of functions.

Communication interfaces 208 may include, for example, wireless chipsets, antennas, wired ports, signal converters, communication protocols, and other hardware and software for interfacing with external systems. For example, device 200 may receive text, audio, executable code, video, digital information or other data via communication interfaces 208 from remote data sources (e.g., remote servers, internet locations, intranet locations, wireless data networks, etc.) or from local media sources (e.g., external drives, memory cards, specialized input systems, wired port connections, wireless terminals, etc.). Example communication networks include Public Switched Telephone Network (PSTN), Public Switched Data Network (PSDN), a short message service (SMS) network, a local-area network (LAN), a voice over IP (VoIP) network, a wide area networks (WAN), a virtual private network (VPN), a campus area network, and the Internet. An example network may communicate through wireless, wired, mechanical, and or optical communication links. Many other communication networks may also be suitable for the embodiments discussed herein.

User-interface 212 may facilitate receiving user-input and user-commands into device 200 and outputting information and prompts for presentation to a user. Although such interfaces typically connect with human users, user-interface 212 may alternatively connect to automated, animal, or other non-human “users.” Additionally, while input and output are described herein as occurring with a user is present, user-interface 212 need not present information to any actual user in order for present functions to be performed. User-input may be received as, for instance, wireless/remote control signals, touch-screen input, actuation of buttons/switches, audio/speech input, motion input, lack of interaction for a predefined time period, and/or other user-interface signals. Information may be presented to the user as, for instance, video, images, audio signals, text, remote device operation, mechanical signals, media file output, etc. In some cases, separate devices may be operated to facilitate user-interface functions.

An example system may also include a variety of devices or elements other than those shown in FIG. 2. For example, device 200 may include visual displays or audio output devices to present results of an example process. As another example, CRM 204 may store computer applications for specific data-generation or data-processing functions. Other examples are possible.

### II. Example Methods

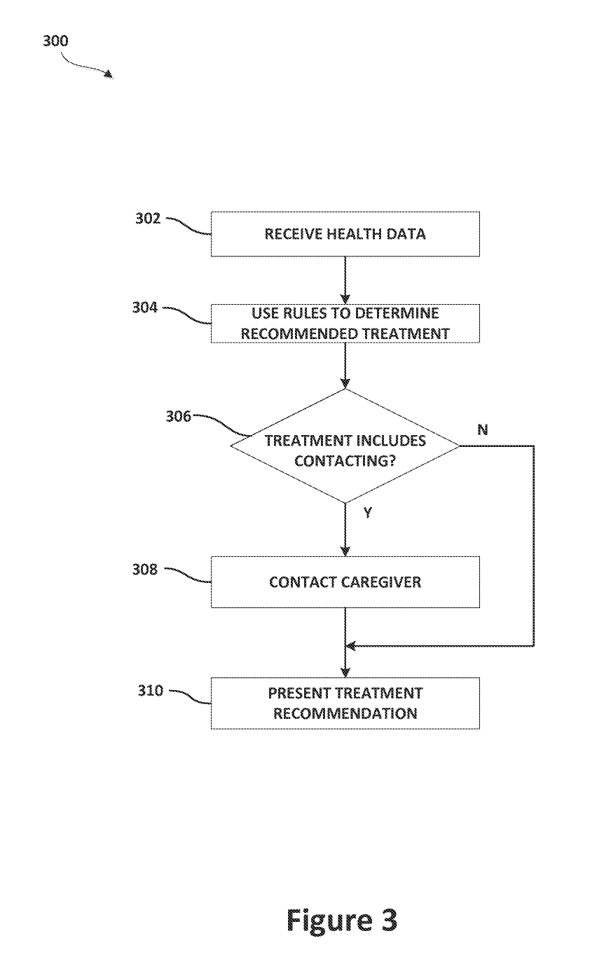

FIG. 3 illustrates a method 300 according to an example embodiment. As shown, method 300 includes receiving health data to a processing system (step 302). Method 300 also includes using preset rules to determine a recommended treatment based on the received health data (step 304). Method 300 further includes making a determination of whether the recommended treatment includes contacting the medical caregiver (step 306). If the medical treatment does not specify contacting the caregiver, then method 300 continues on to step 310. If the recommended treatment does include an instruction to contact the caregiver, then method 300 involves the processing system automatically sending a notification request associated with the identified caregiver (step 308). Method 300 further includes presenting the recommended treatment to either the contacted caregiver or another staff member (step 310).

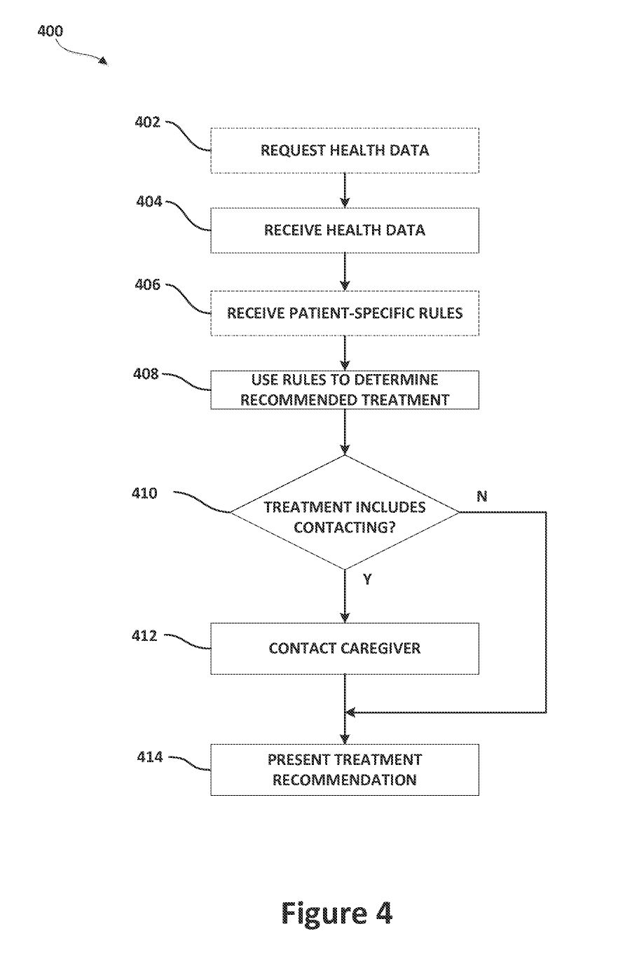

FIG. 4 illustrates a method 400 according to an example embodiment. As shown, method 400 includes steps 404 and 408-414, which directly correlate with the steps in method 300. Additionally, method 400 involves the steps of requesting health data (step 402), and receiving patient-specific rules for inclusion in the treatment protocols associated with the patient (step 406).

Regarding step 402 of method 400, the processing system may request health data in any of various ways. In some cases, the system may request data on a periodic basis, in order to keep a patient constantly monitored. In other cases, the system may request data in response to specific settings or inputs. For example, the system may receive user-input requesting treatment recommendations and responsively request health data from the data sources. As another example, the processing system may be programmed to request certain health data from certain monitor sources in response to detecting a particular health condition from other monitors or input sources. In some embodiments, a request for health data may be submitted as a data message readable by a processor or user associated with medical equipment. In other embodiments, the request for health data may be executable instructions that cause the medical monitoring system to automatically collect the health data. Data need not always be requested specifically from medical monitoring systems. For instance, external memory for networked devices may also be queried for information regarding a patient. As another example, medical monitors may be configured to provide new data without request.

As shown at steps 302 of method 300 and 404 of method 400, an exemplary method may involve receiving health data from one or more sources. In some cases, as discussed above, the data may be received in response to a request from the processing system to the medical monitor or external source. In other cases, components and the external source may indicate that data should be transmitted to the processing system. In still other cases, an agent type software may be programmed to recognize new data associated with a particular patient and automatically forward the received data to the processing system.

Received health data may include many different types of information. In some cases, information may be qualitative, such as medical personnel, professional opinions, personal medical history data, family history information, or general information regarding particular medical conditions. Such qualitative data may be analyzed by the system for keywords or recognizable prognoses, so that the system may assign its own quantitative values. Quantitative values may be any of various data structures, for example, Boolean operators, numerical data, scored text data, integer data, relative ranking data on mathematical function data. Examples of health data that can be used as metrics in such a system are too numerous to be reasonably recounted here. A person of skill in the art would recognize that any known type of lab result, previously-known patient information, or monitored vital signs could be used as quantitative health data. In some protocols, a value of interest may be the definite or current value of a particular metric. For example, a threshold may be set at a lower limit of fraction of inspired oxygen (FIO2), and anything below that amount would cause an alert. In other cases, the dynamics of the metric may be the value of interest. For example, a threshold may be set to alert a caregiver when the oxygen saturation of a patient is increasing too quickly. Many other examples could be used.

With regard to requesting and receiving health the protocol information to the processing system, not all data must be received from external sources. For example, some user interfaces or medical equipment may be integrated directly into the processing system. In such an embodiment, the health data and protocol information may be received internally at a processor in the system, rather than externally received at the processing system is a whole.

As shown at step 304 of method 300 and step 408 of method 400, an exemplary embodiment may include using pre-specified treatment rules to determine a recommended treatment for a patient. In an exemplary embodiment, the recommended treatment may be based on the received health data and/or other health data stored at the system. In this disclosure, the full set of rules that can be applied in a given process is referred to as the treatment protocols. Treatment protocols may include any type of logical mathematical for decision-making structures for taking health data is an input and outputting one or more recommended treatments. For example, a rule configured to receive the current value of a particular vital sign as input may associate each of several treatment actions with each of several levels of the particular vital sign. As a particular example, a rule that uses a Boolean decision-process may define one treatment for values below a certain threshold and one treatment for values above a certain threshold. As another example, a rule may define the optimal mathematical relationship between a set of health factors and an amount of treatment. More complex decision-making structures may be defined similarly, with combinations of multi-leveled metrics defining a wide range of treatment actions and amounts.

In some embodiments, a general set of rules may be defined for a given type of patient (e.g., all patients with a certain condition have the same protocol by default). Additionally, as shown at step 406 of method 400, an example method may include receiving patient-specific rules to be used in determining a recommended treatment. For example, a physician may change the default threshold levels for partial pressure of carbon dioxide (PCO2) in arterial blood of a patient with a naturally high baseline PCO2. As another example, if a physician is worried about a particularly unstable patient, the physician may set a patient-specific rule to be contacted in certain extreme conditions that would normally be handled by an intern. As a physician or medical team becomes more experienced with the treatment recommendation program, authorize users may alter the default protocols to suit their experience. Additionally, authorized users may define patient-specific profiles, which modify the default protocol in specified ways. For example, if all diabetic patients have a specific difference in their vital signs or risk factors, regardless of any current prognosis, the physician may define a “Diabetic Patient” profile, that modifies the protocols of any regimen to compensate for the specific difference associated with diabetes. Many other profile types may be created to modify the default protocols in a system. Once a set of profiles have been defined a given patient may receive their own patient-specific protocols used on the default protocol and the various profiles that match their particular conditions.

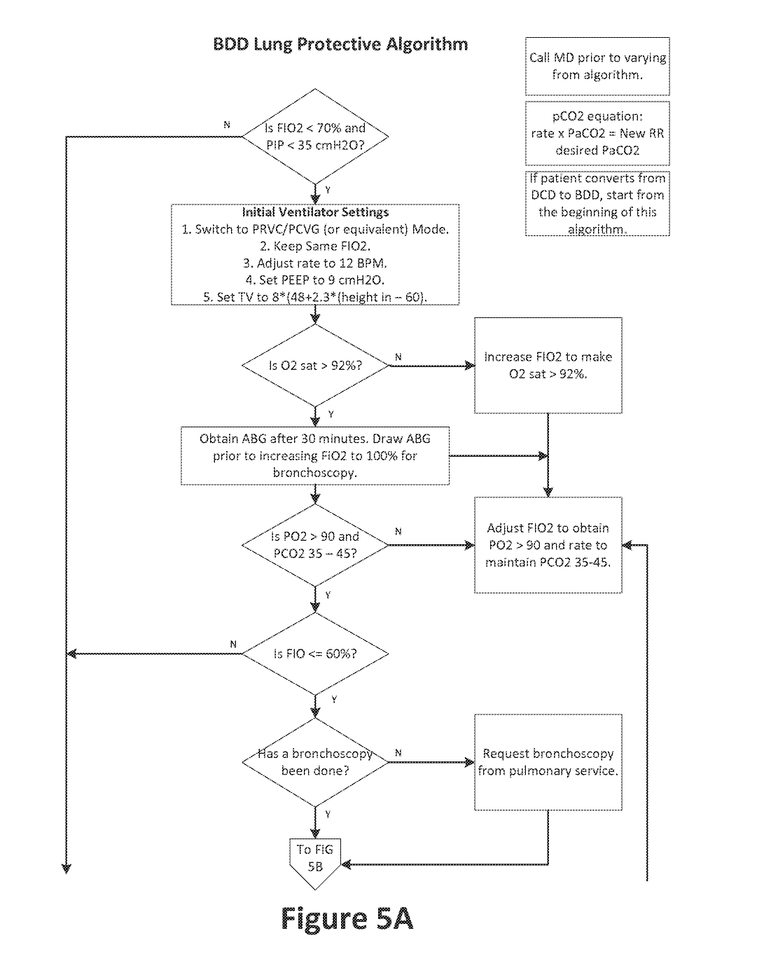

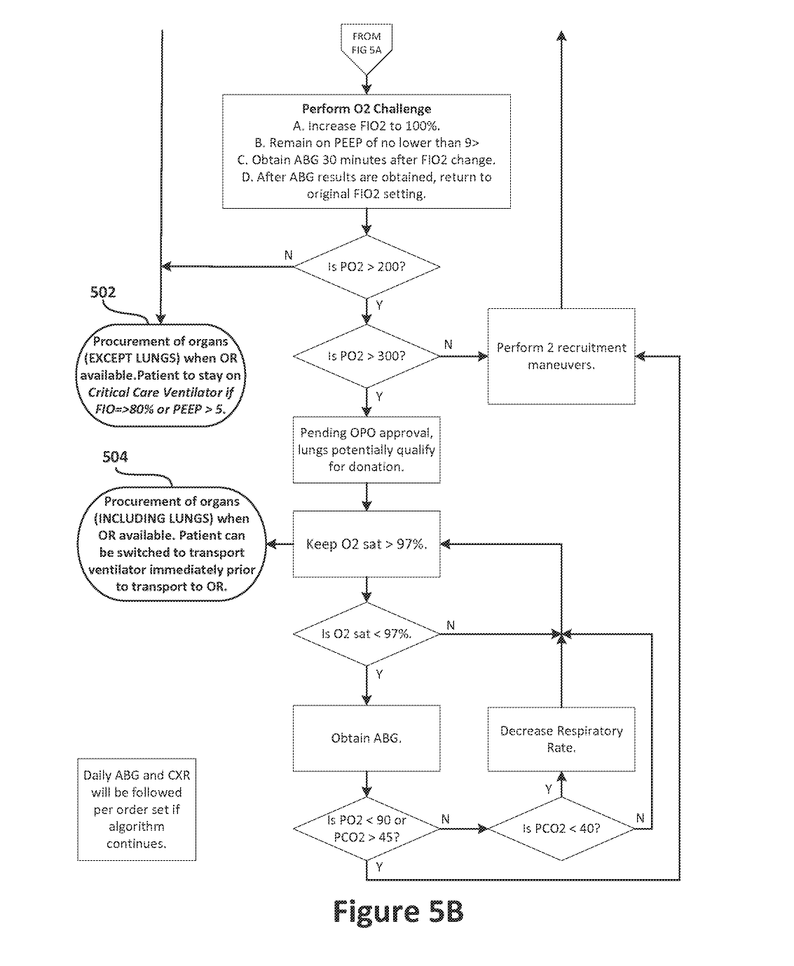

In some cases, physicians may define patient-specific rules that are not based on any default procedure profile. For example, the system may provide a user-interface in which a physician may enter a set of new rules specifically for a particular patient. The user-interface may present the instructions as, for example, a flow chart (similar to those shown in FIGS. 5A-8), a list of potential conditions and a corresponding list of instructions for an attending caregiver, a set of patient-specific questions to be answered, or space for less-structured comments to be displayed whenever a caregiver treats the patient. For structured rules and procedures, the patient-specific rules may be triggered by predefined conditions or diagnoses (e.g., results of lab testing, a time limit, a predefined result of caregiver inspection, a change in vital signs). In this way, the new procedures may integrate into the existing rules and procedures seamlessly. Even for unstructured comments, the inclusion of physician comments in the standard instruction format may improve caregiver compliance and understanding of physician instructions.

Although a physician may specify some particular rules for a certain patient, the system may also detect that certain rules are not affected by the new rules and, responsively, maintain the unchanged rules in effect. For example, if procedures specify that a certain blood-pressure profile should produce an alarm, and a physician only specifies patient-specific rules regarding heart rate, then the system may continue to use the blood-pressure rule in combination with all other rules for a patient. In some cases, the system may be configured to query the physician as to whether a preexisting rule should still be enforced at the time the physician specifies new rules.

In addition to patient-specific profiles and rules, authorized personnel may also create physician-specific profiles to match the preferences of the particular physician or situation. For example, one physician may wish to be contacted for certain sets of vital signs, while another physician treat those same vital signs as a condition that can be handled by a medical intern. As another example, physicians may wish to have a preprogrammed profile to use when the medical intern or other caregiver on duty is new or inexperienced with a particular protocol (i.e., an “Inexperienced” profile may specify that the physician is contacted for more routine procedures than normal or that instructions are more explicit). In some cases, a physician-specific protocol may be automatically assigned by the system in response to detecting that a particular physician or intern is assigned to the patient. For example, the system may store different instructions for each of the interns assigned to a patient without the physician needing to specify the instructions personally.

In some implementations, it may be reasonable to provide only a single recommended treatment. In other cases, it may be preferable to present a list of potential treatments along with information regarding the benefits and risks associated in each of the options. In practice, a single “treatment action” may actually define a set of individual changes or interventions. For example, in a multiple-input treatment regimen, a single treatment recommendation may include changing each of the various inputs for a combined effect or to avoid undesirable interactions (e.g., some drugs become toxic when combined).

Treatment options may vary greatly based on the application. For example, some recommended treatments may specify that no change for intervention need be made. If medical personnel have requested a treatment recommendation, and the protocols returned the recommendation of no change, then such recommendation may be presented on a display screen to the medical personnel. In contrast, if the recommendation of no change is determined from an automated or periodic recommendation request, then the system may respond by refraining from taking action for contacting medical personnel.

In other cases, the system may recommend a change in treatment or intervention for the patient. For example, a change in treatment may be a change in the value of a current treatment (e.g. medicine volume or concentration, beat rate for a heart machine, breath rate for a lung machine, etc.), a recommendation to add a new treatment for intervention to a patient's current regimen, or a recommendation to cease a particular intervention.

In an example treatment protocol, some recommended treatments may include a recommendation to contact a medical caregiver. For example, some treatment changes require a nursing professional or medical intern to perform the actual changing. For such a recommended treatment, the protocols may define an urgency of the treatment change, so that the system may determine whether or not to call a caregiver immediately. In particular, an urgency value may define a maximum amount of time that the patient may remain safe with the current treatment regimen. Therefore, if the health data was determined through an automated process, rather than being requested by a caregiver, then the system may determine a latest safe time to contact the caregiver. If the treatment change is critically urgent, then a safe amount of time would be specified as zero and the caregiver would be immediately contacted. If, instead, the system determines that the treatment is safe to postponed, then the system may wait to send a notification request until either the caregiver comes independently (e.g., during periodic rounds), or the amount of time runs out. If the safe amount of time runs out before the caregiver checks on the patient, then the system may send a notification request. Some recommended treatments may not require contacting the caregiver but may be applied during the caregiver's next periodic rounds.

As an alternative to the “amount of time” implementation of an urgency value, some embodiments may specify other changes based on the determined urgency. In particular, the system may send out a low-urgency notification immediately, but may send it out differently than high-urgency messages are sent. For example, instead of a high-importance medium (e.g., hospital intercom, direct phone call) the system may use a low urgency medium (e.g., message to a pager, a text message specifying that the treatment is low urgency, etc.) for a low urgency notification. As another example, a low-urgency notification may be sent through only a single network, while a high-urgency notification is sent through multiple media to ensure delivery.

Some treatment recommendations may require a physician to personally determine an appropriate treatment, rather than simply using rules. In such a situation, the recommended treatment may be to contact the physician. In some situations, a recommended treatment procedure (e.g., surgery, organ extraction, resuscitation, etc.) may require presence of both the physician and other medical staff. Accordingly, such a recommendation may be programmed to cause the system to contact both the medical staff and the physician.

When the system determines that a caregiver (e.g., a nurse, intern, or supervising physician) should be called to carry out or determine a recommended treatment, the notification may be sent out automatically to a notification system. As shown in FIG. 1, the notification system may connect processing system 102 with external devices 104 and/or intercom 106. In some embodiments, the notification system may be integrated within processing system 102. In other cases, the message may be sent from a local or remote system to instruct the notification system to facilitate contact with the caregiver. The identified caregiver may be a particular nurse, intern, doctor, surgeon, etc., or the caregiver may be anyone from a set of caregivers that match a general profile. For example, if the rules specify that a resident should be contacted in a particular situation, the process may send out the notification request and the notification system may contact one or all of the residents in response to the message. Alternatively, the processing system itself may access lists of caregivers and determine whom to contact independent of the notification system.

The notification request may be readable data or an executable file. If readable data is sent, then the notification system may determine the specifics of the notification and generate the message (in some cases with a user reading the message and others with a machine automatically reading the message). The message may be sent out either over the loudspeaker of the intercom 106 or through a communication network to a mobile computing device 104 associated with the caregiver. For this reason, a database of contact information for attending caregivers may be saved within the system or in external memory. In some instances, the notification may be created and transferred to the communication network or intercom without requiring any human intervention. The content of the notification that is presented to the caregiver may include any type of information that is deemed necessary for the caregiver. Some examples include patient name, urgency level, recommended treatment, patient location (perhaps including a hospital map), needed supplies, other members of the medical team, among other examples.

Once a caregiver responds to a notification or requests a treatment recommendation during normal patient checks, the system may present the recommended treatment to the caregiver. In some cases, additional questions may be asked of the caregiver to refine the treatment recommendation before presenting the recommendation to the caregiver. The recommendation may be presented, for example, on local display device, on an integrated display device, in audio signals, or through tactile communication. In some implementations, the treatment may be presented in a step-by-step format to help ensure that the full process is completed. In other cases, the recommendation may be presented in a single message. In either case, the system may monitor patient vitals in order to assess the effectiveness of the treatment.

### III. Protocol Examples

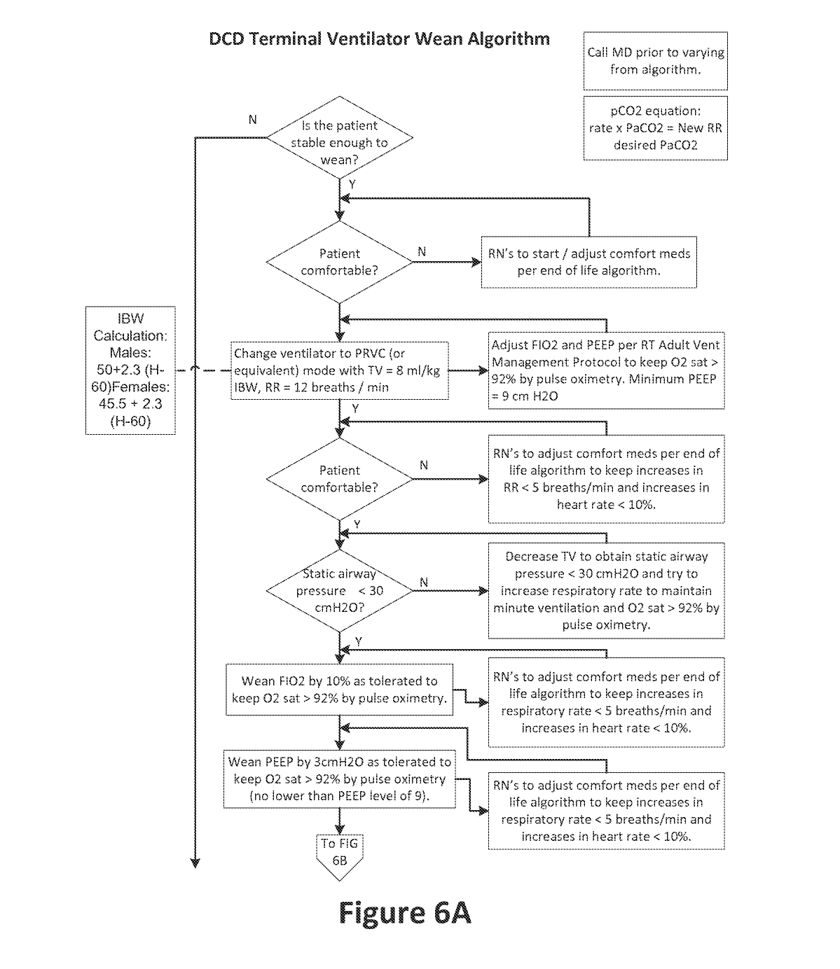

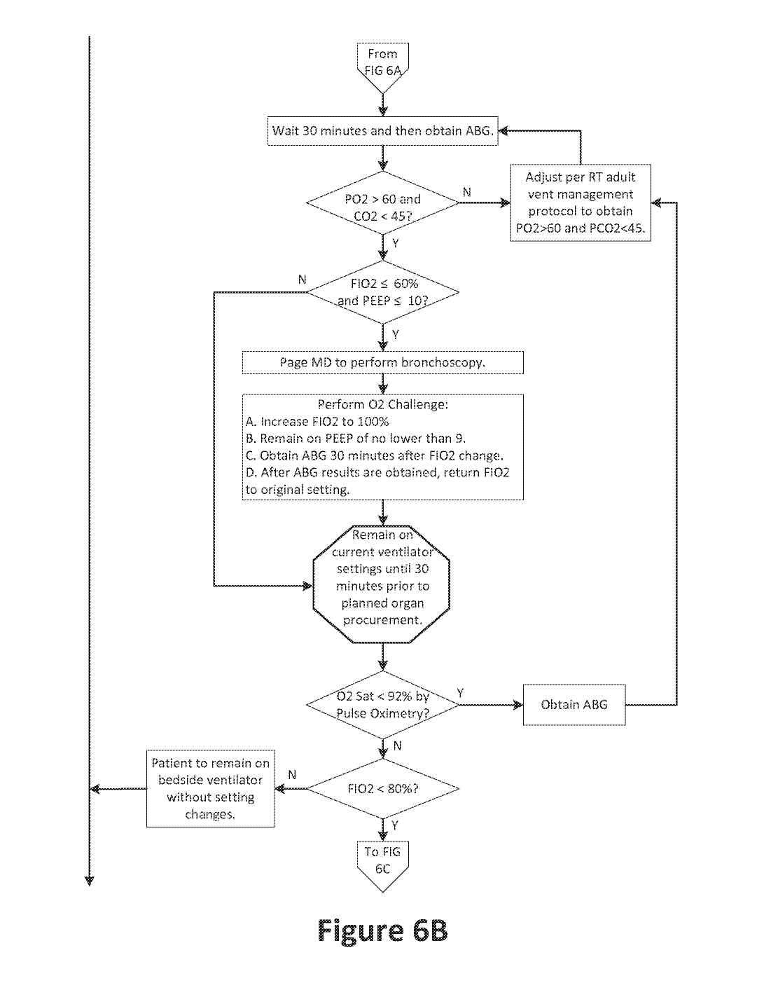

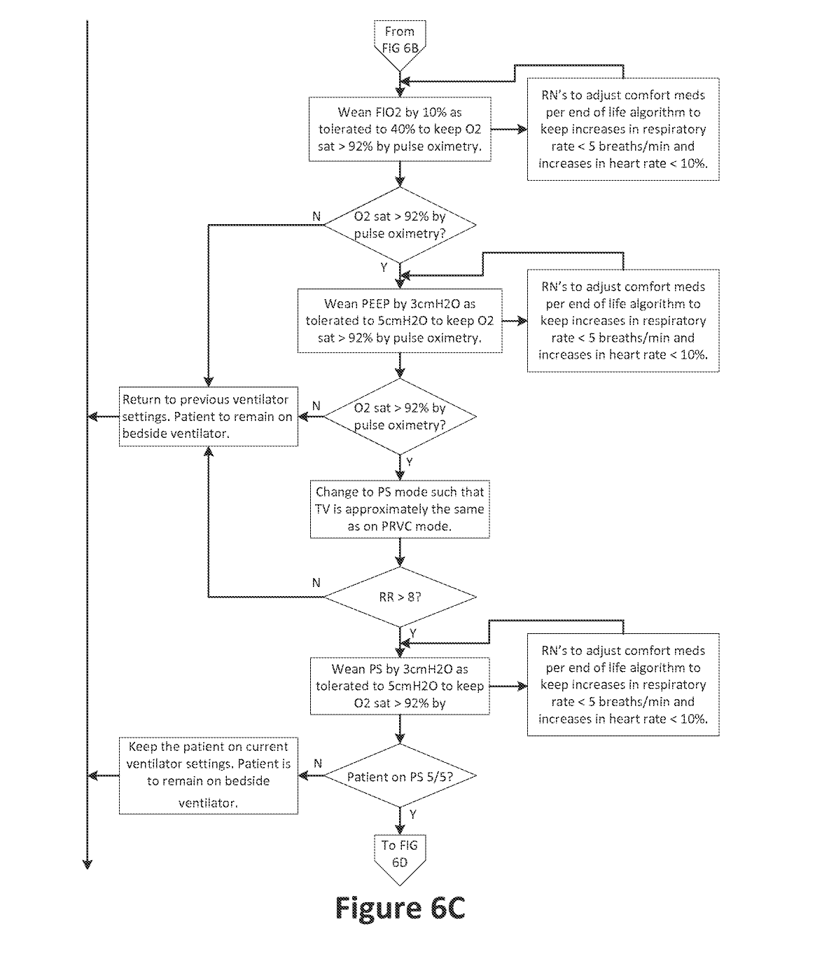

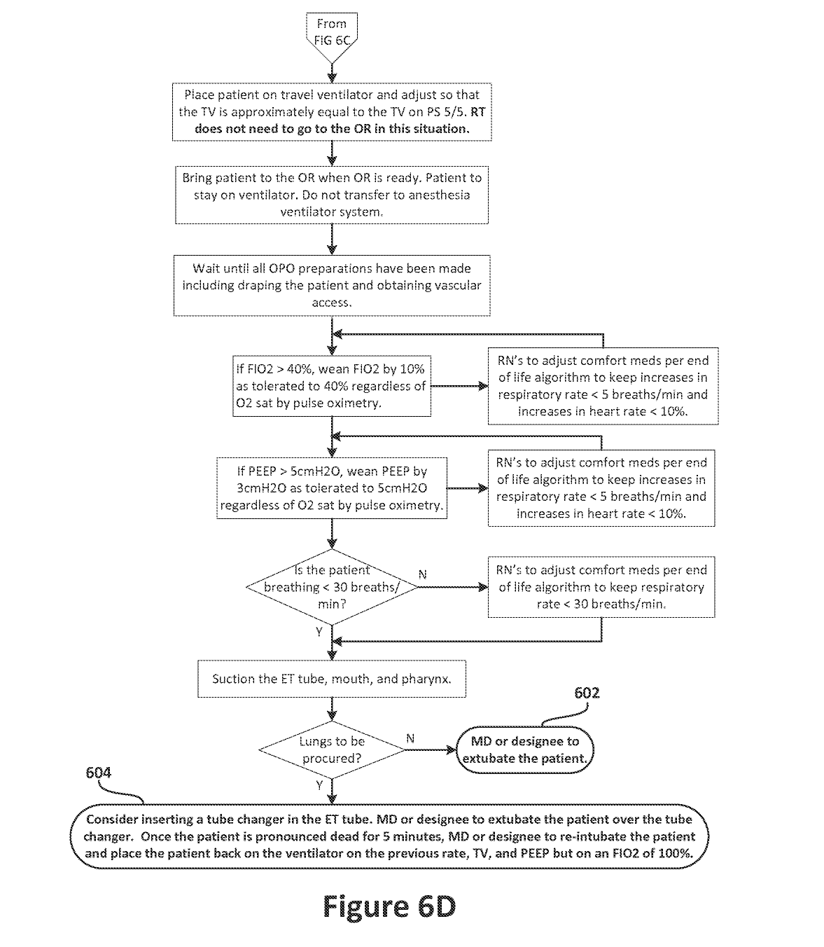

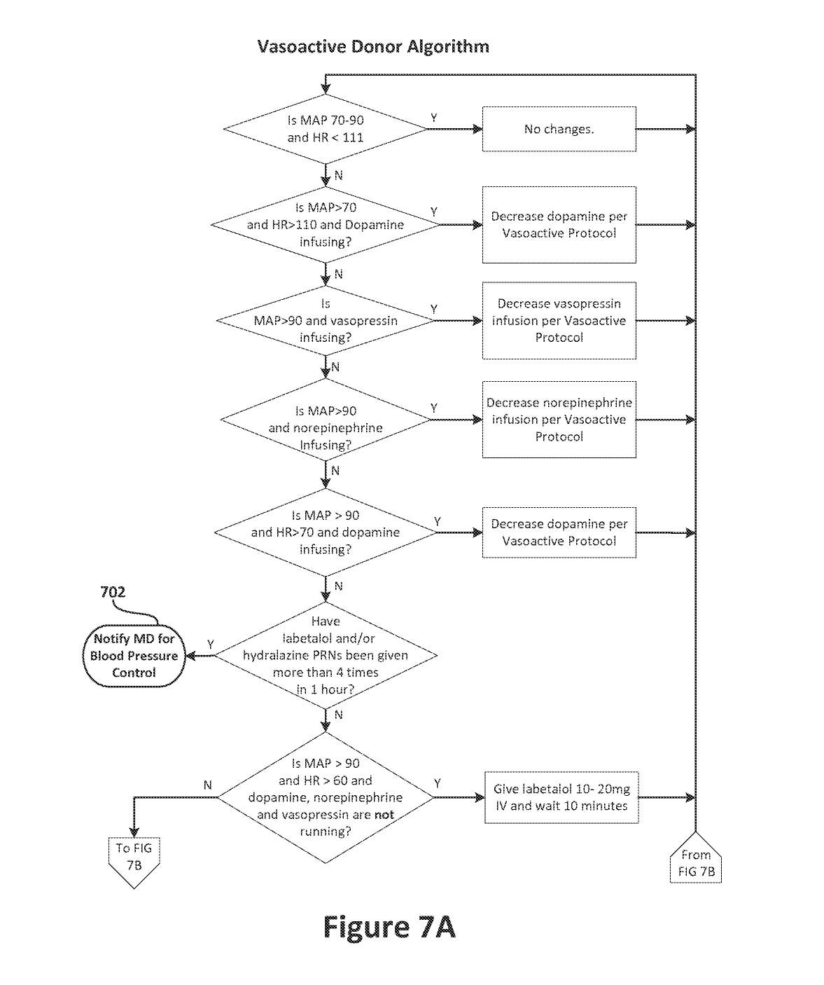

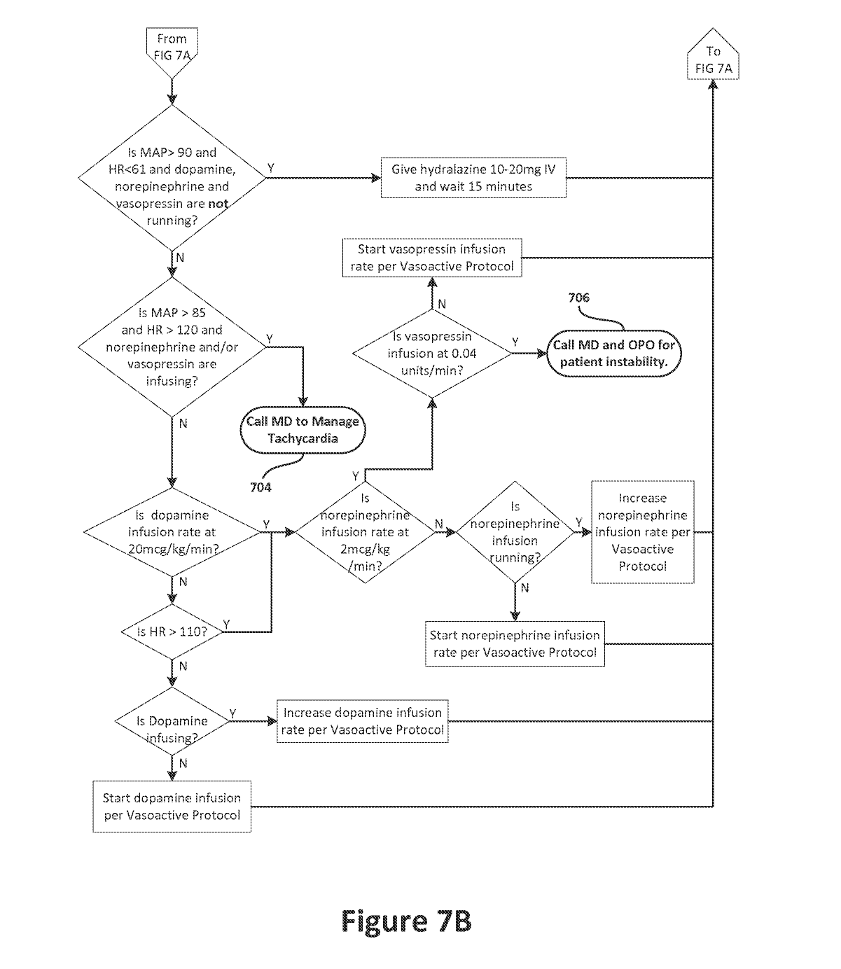

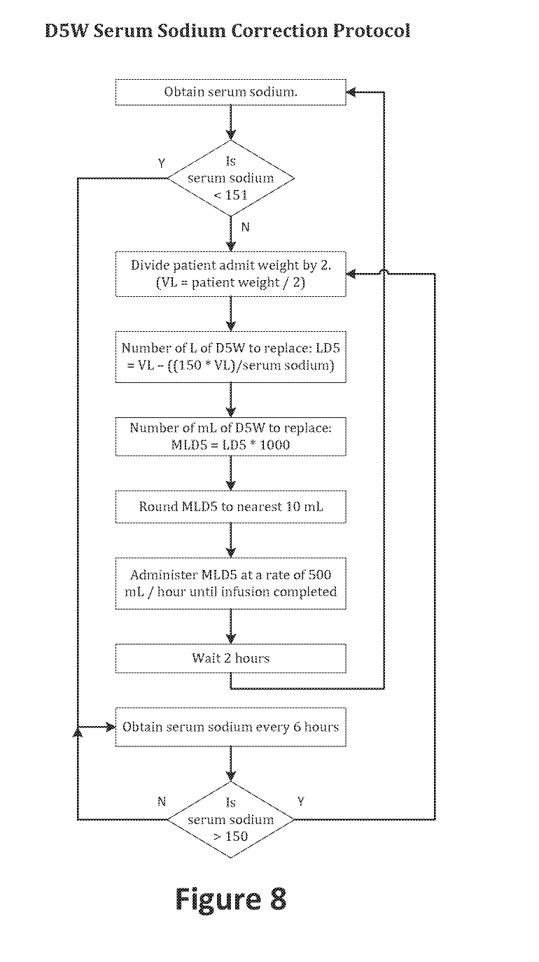

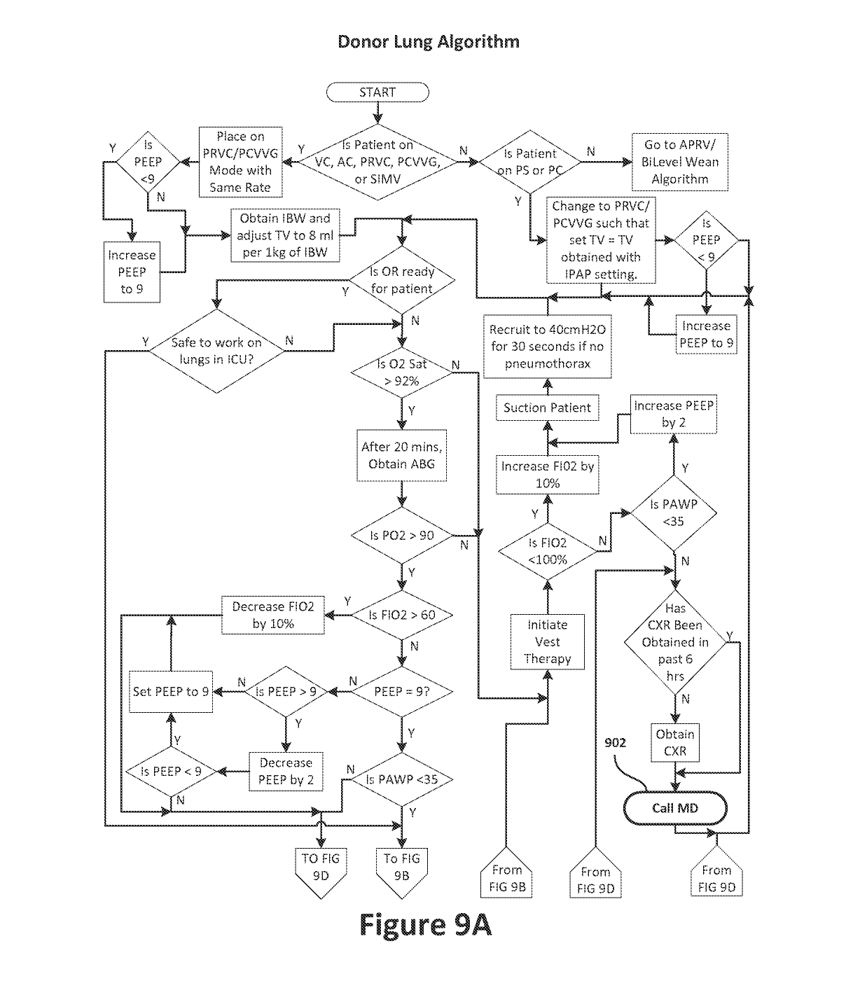

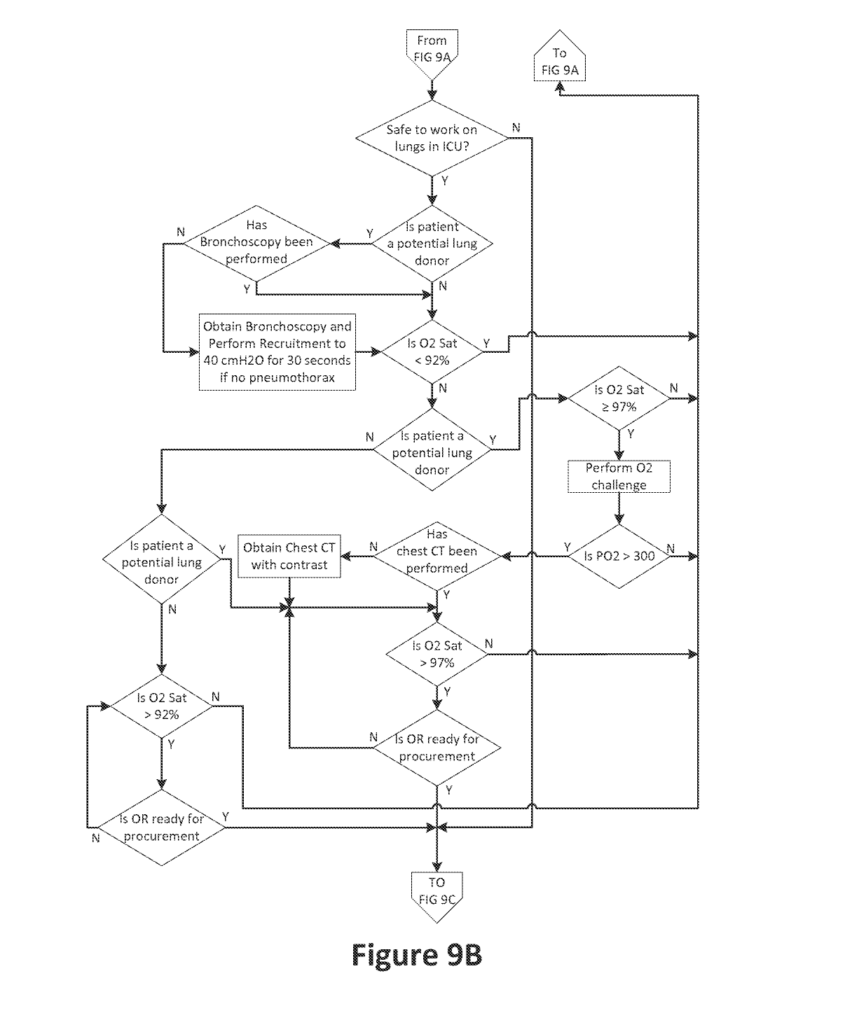

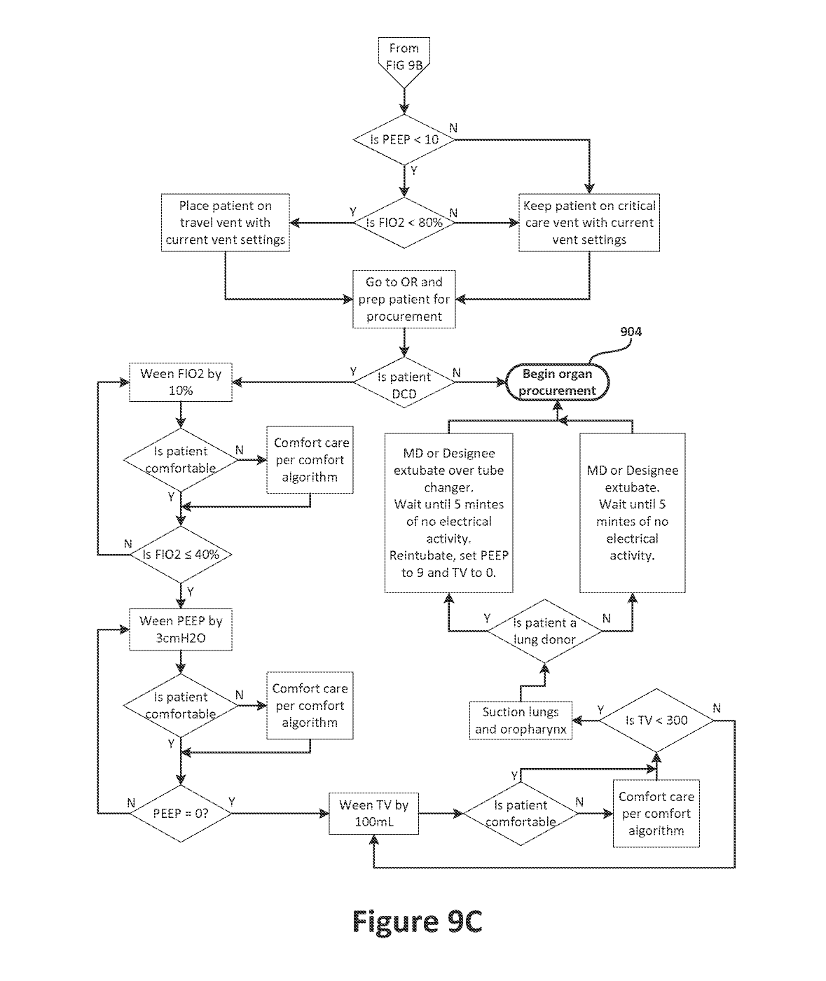

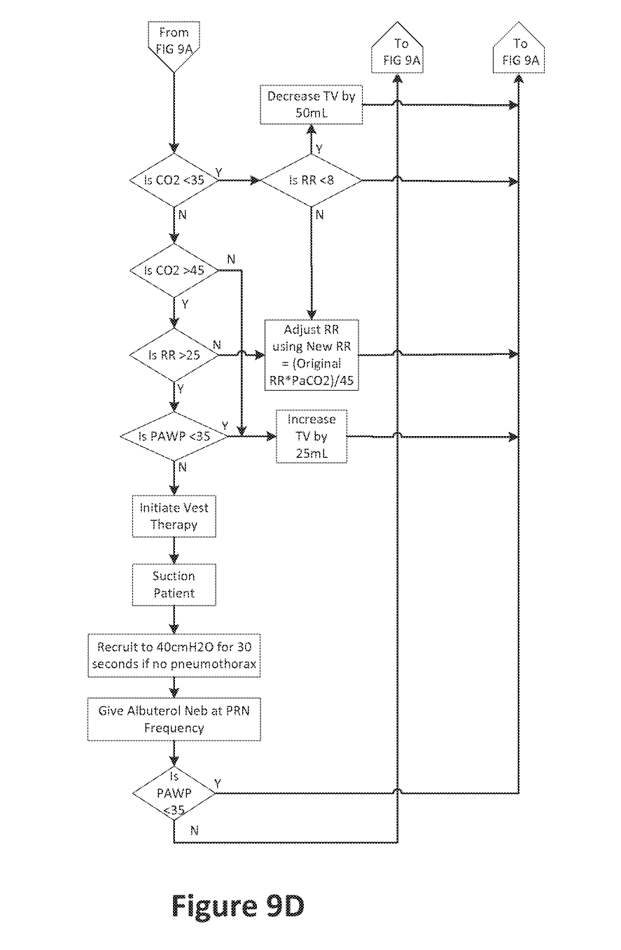

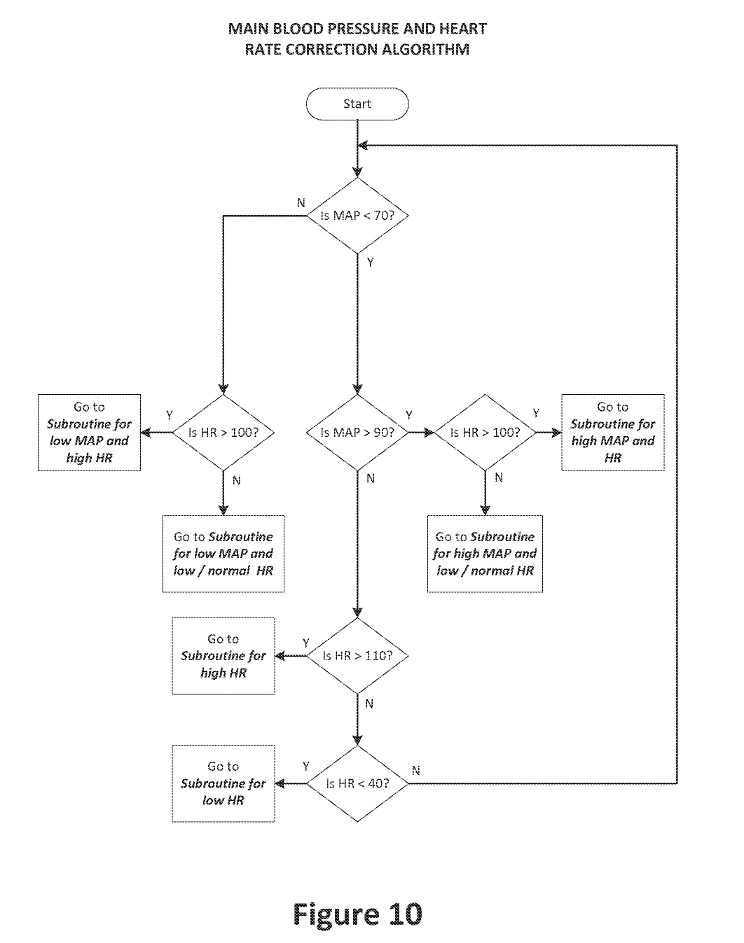

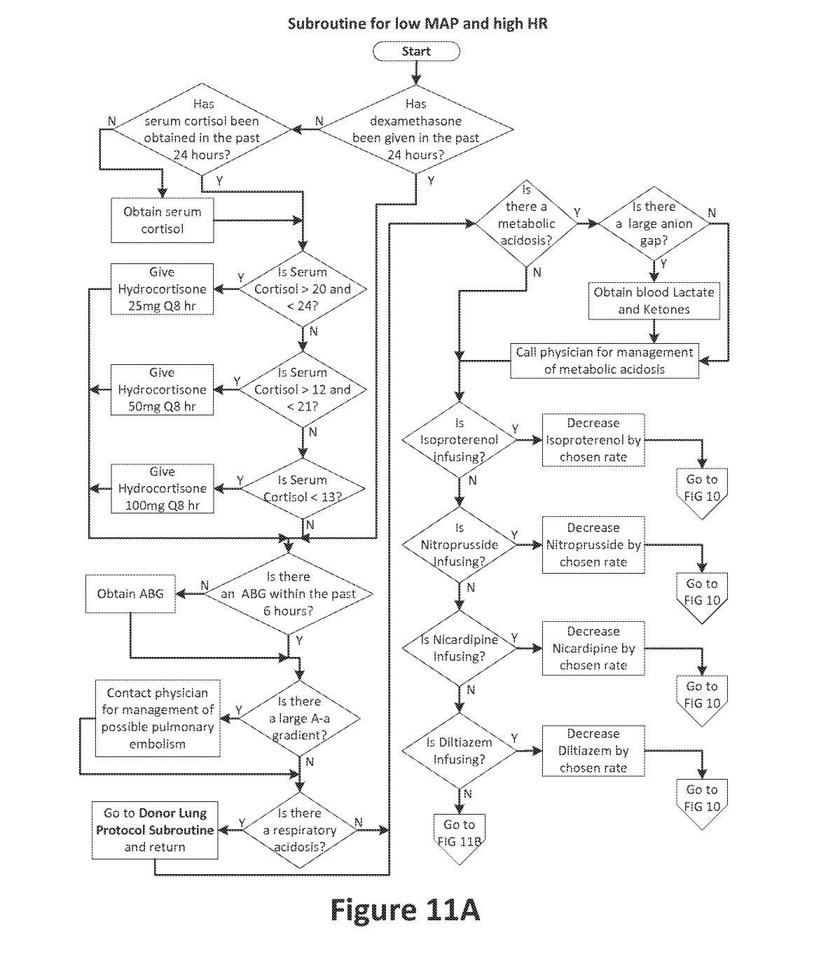

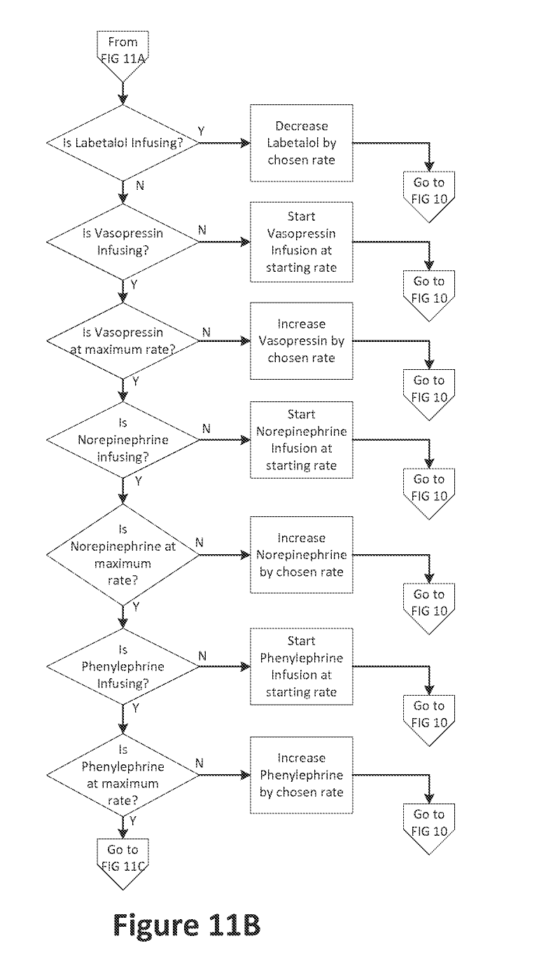

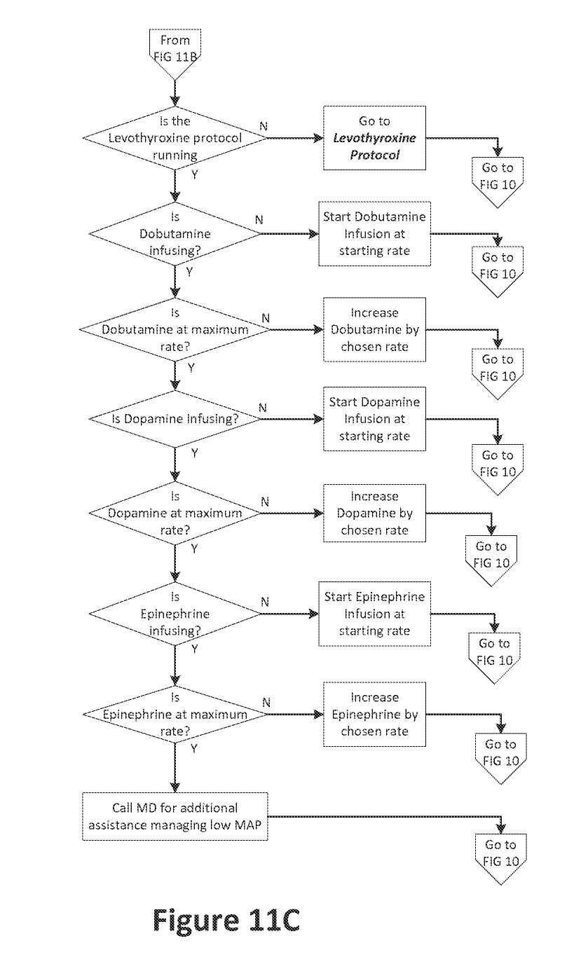

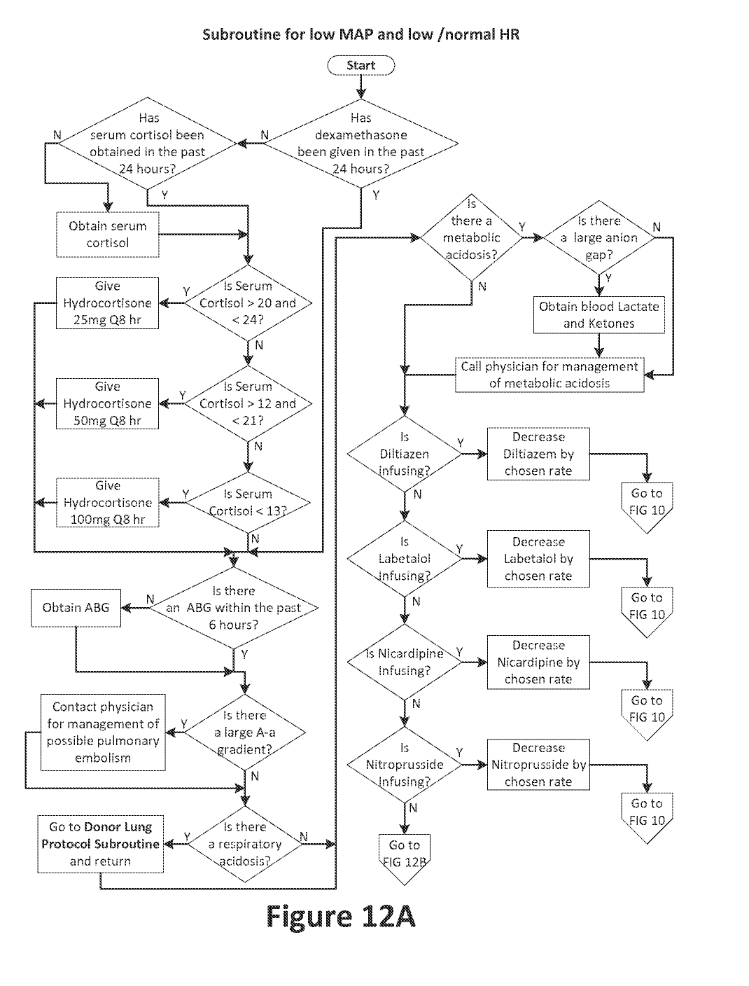

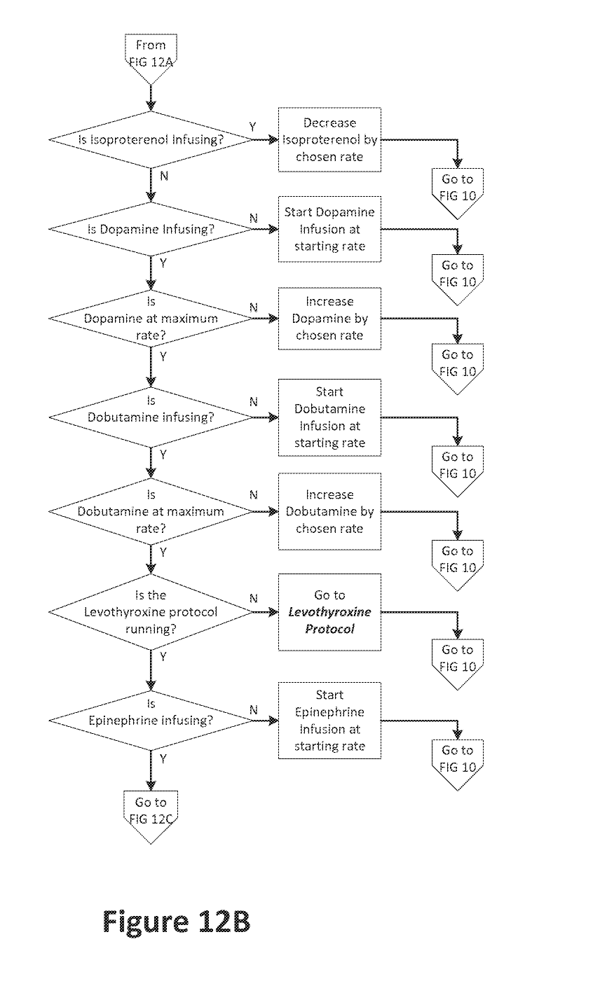

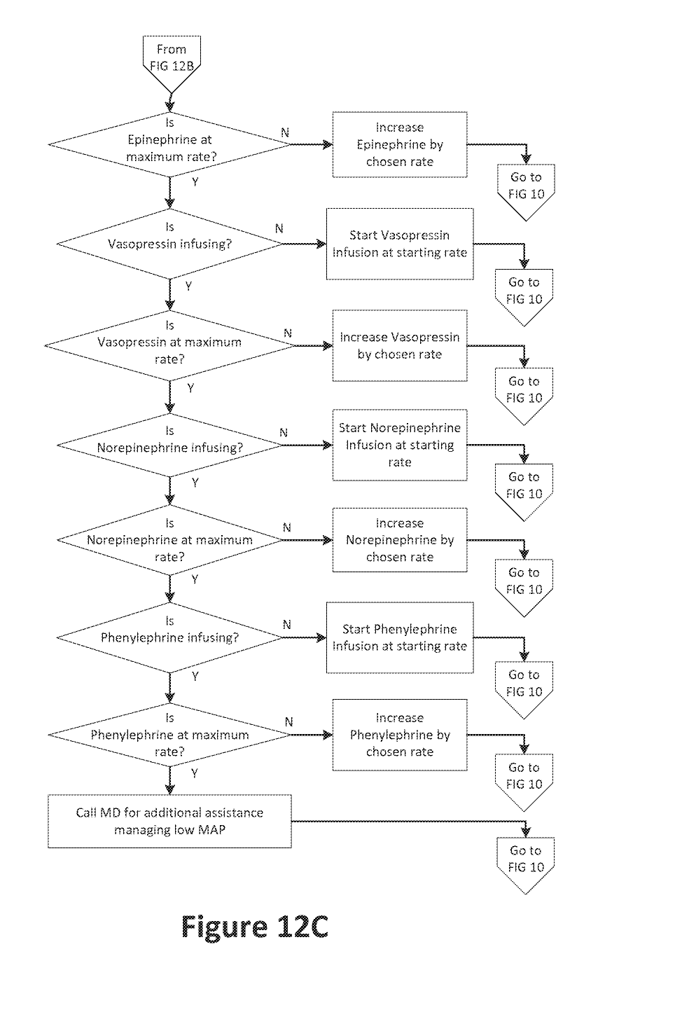

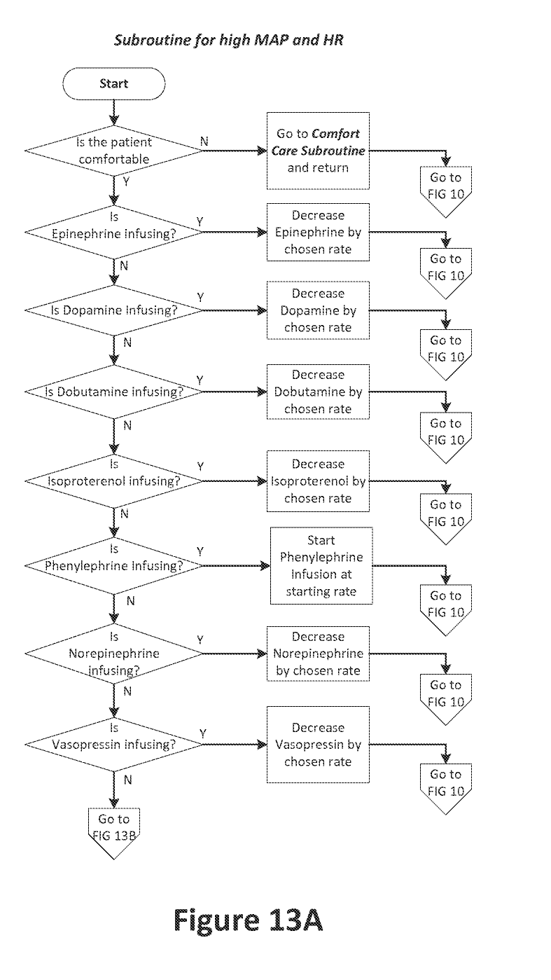

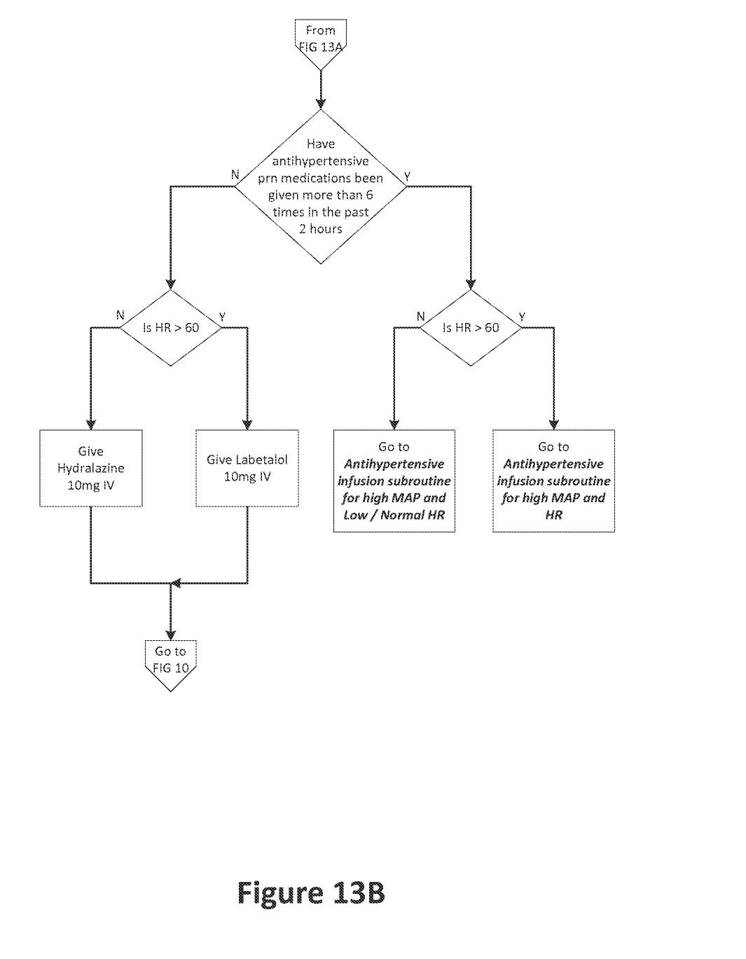

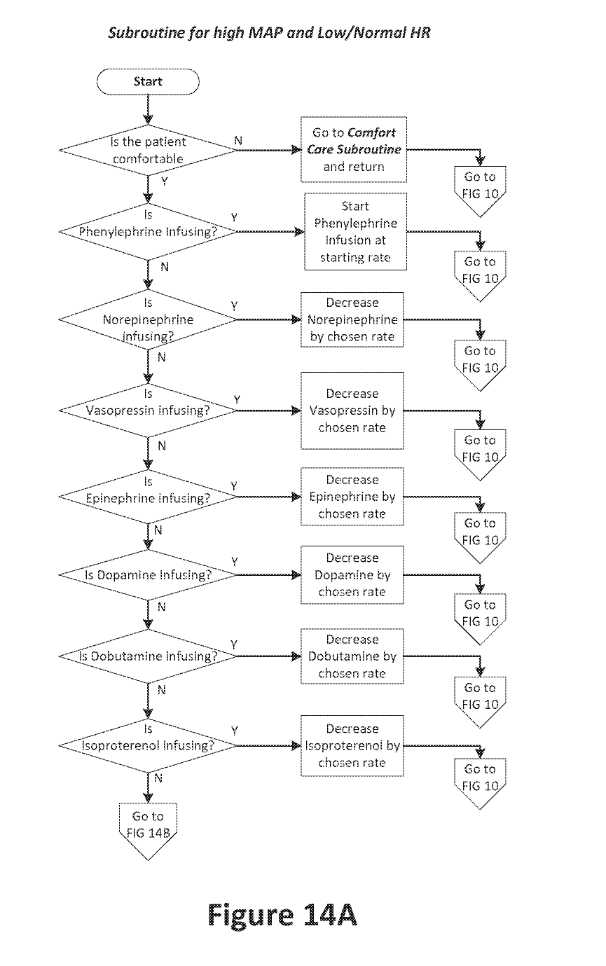

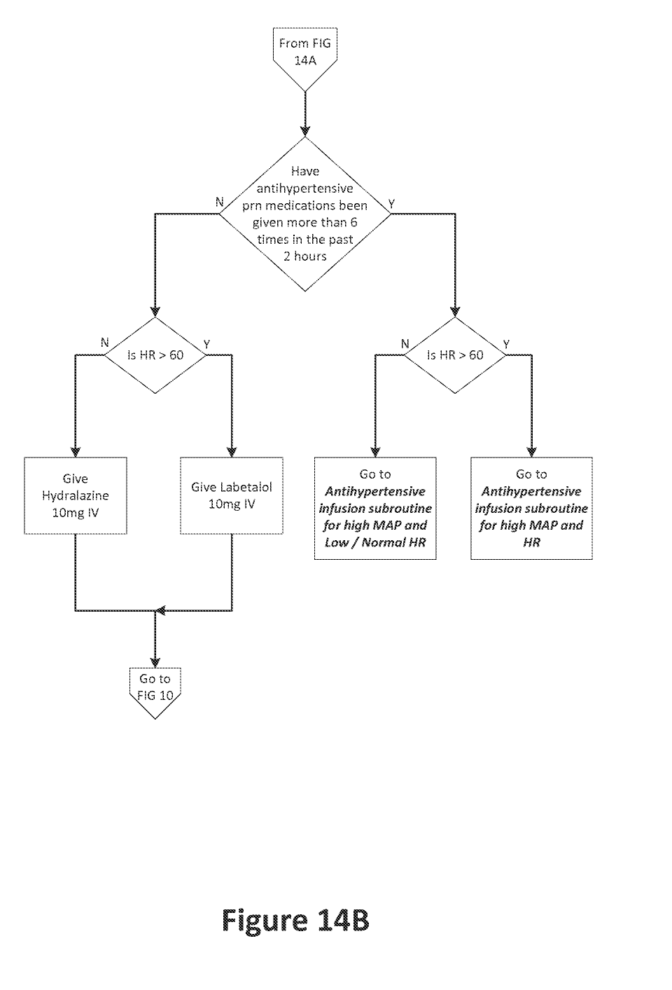

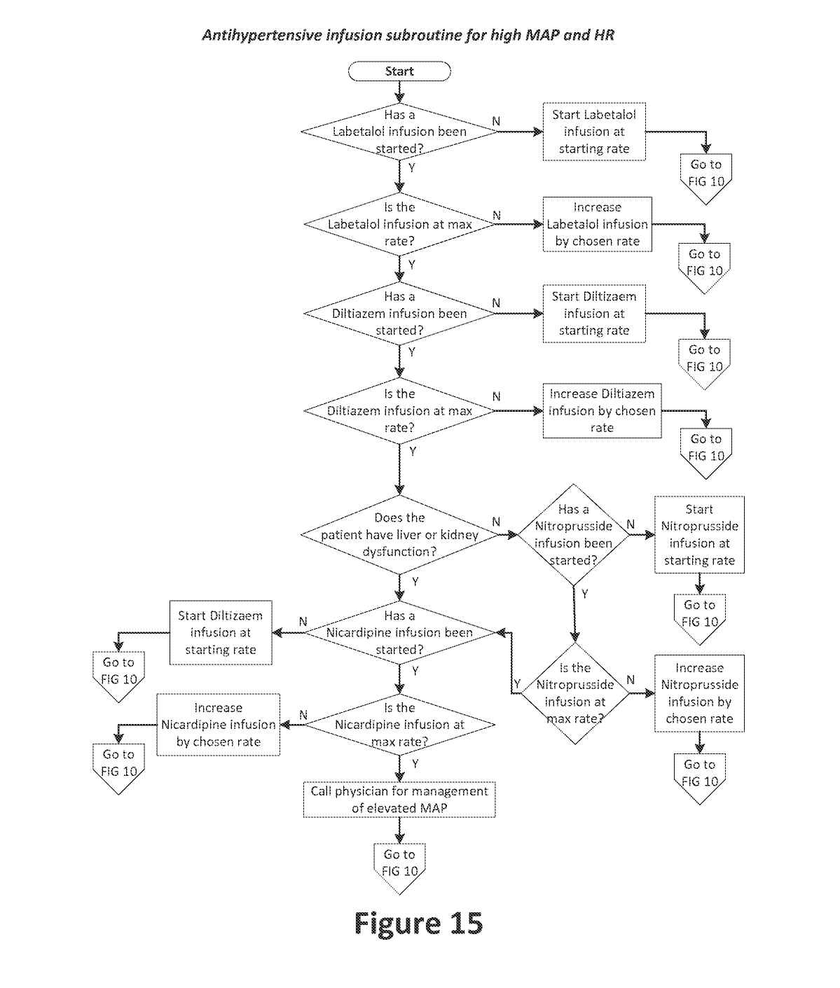

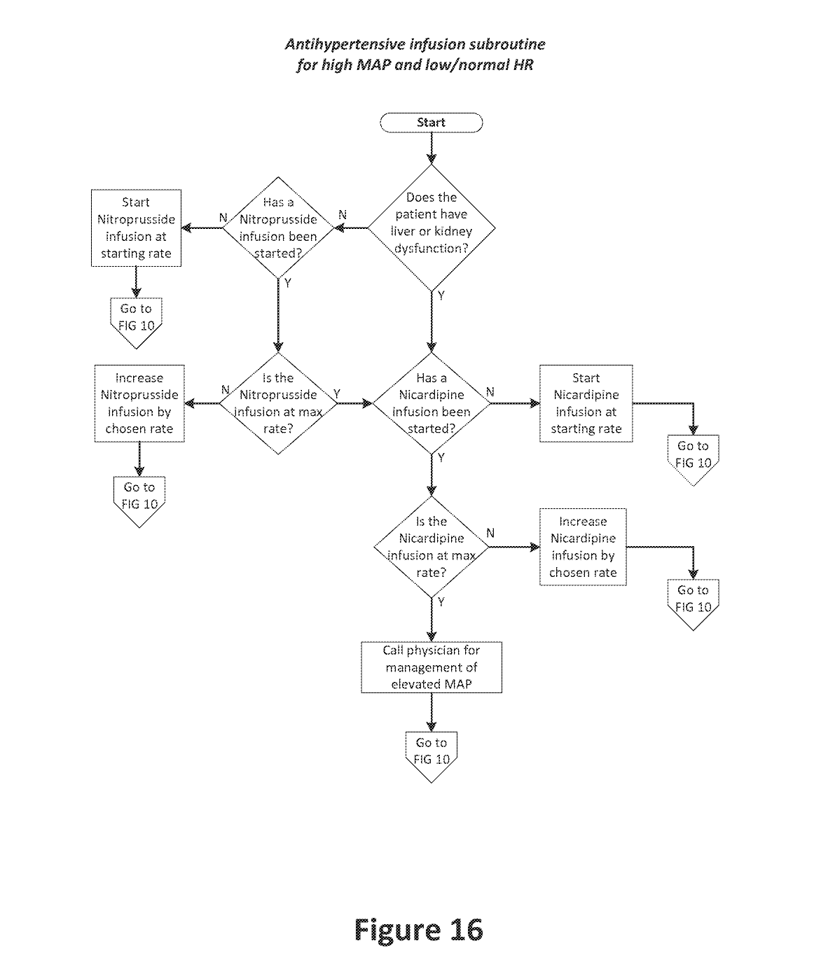

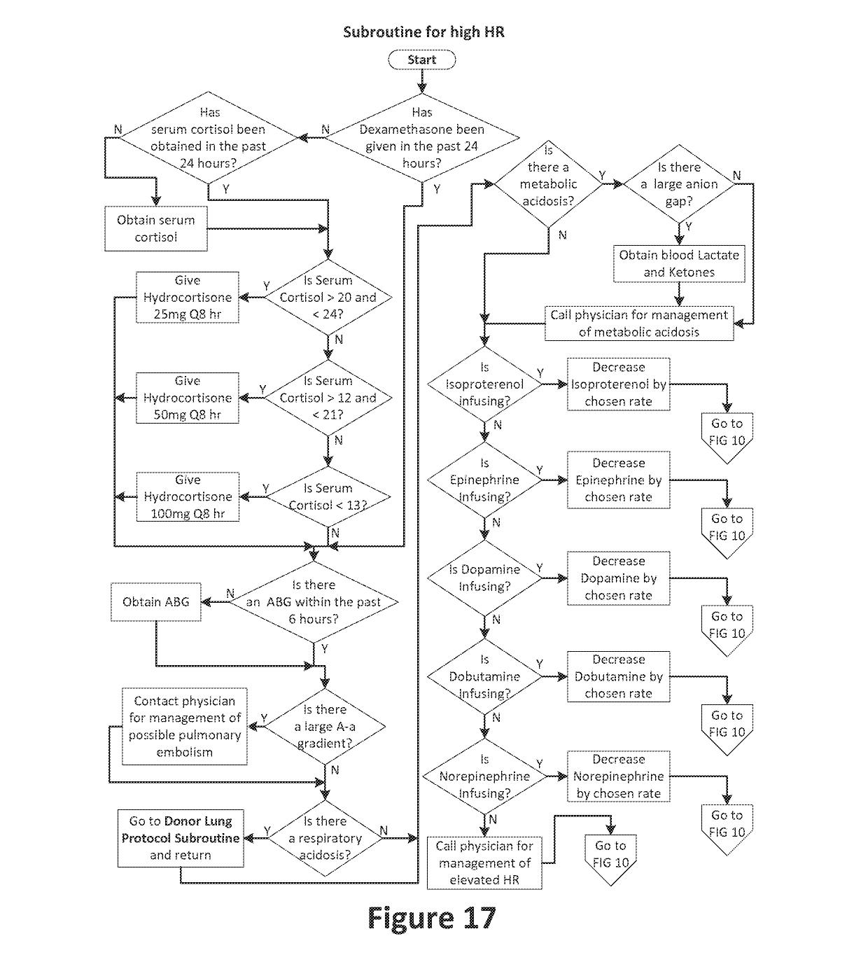

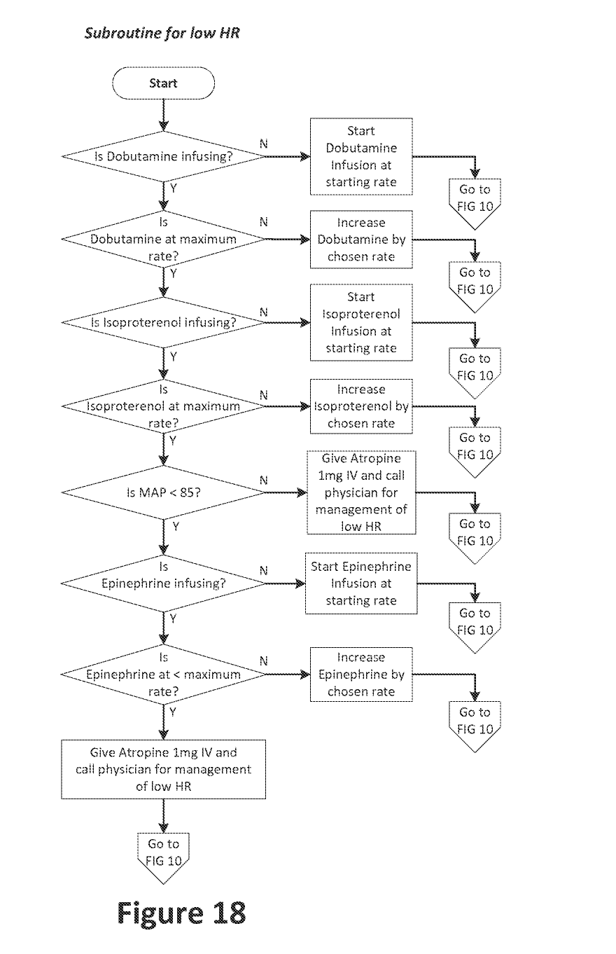

FIGS. 5A through 18 show particular example protocols that may be implemented using example methods, system s, or media. In particular, FIGS. 5A and 5B show example procedures for a Brain-Dead Donor Lung Protective Algorithm, FIGS. 6A-6D show example procedures for a Donation after Cardiac Death Terminal Ventilator Wean Algorithm, FIGS. 7A and 7B show example procedures for a Vasoactive Donor Algorithm, FIG. 8 shows example procedures for a D5W Serum Sodium Correction Protocol, FIGS. 9A-9D show example procedures for a full Donor Lung Algorithm, and FIGS. 10-18 show example procedures for a Blood Pressure and Heart Rate Correction Algorithm (main routine on FIG. 10, and subroutines on FIGS. 11A-18).

These examples are given for illustration and should not be seen as necessarily limiting the scope of the disclosed embodiments. As shown, the protocols involve several rules visualized as decision boxes and various treatment procedures visualized by action squares and rounded rectangles. Although threshold and treatment values are shown, these are not the only values that could be used in this system. The values and procedures given, however, do provide particular preferred embodiments that may have features which are desirable or unexpected in practice. In these examples, the following acronyms are used:

ABG: Arterial Blood Gas

APRV: Airway Pressure Release Ventilation

BDD: Brain-Dead Donor

BPM: Breaths per minute

CVP: Central Venous Pressure

CXR: Chest Radiograph (X-Ray)

D5W: 5% Dextrose in Water solution

DCD: Donor/Donation after Cardiac Death

ET: Endotracheal Tube

FIO2 or FIO: Fraction of Inspired Oxygen

HR: Heart Rate

IBW: Ideal Body Weight

MAP: Mean Arterial Pressure

MD: Medical Doctor (physician)

O2 sat: Percent Oxygen Saturation

OPO: Organ Procurement Operation

OR: Operating Room

PAWP: Pulmonary Artery Wedge Pressure.

PEEP: Positive End-Expiratory Pressure

PIP: Peak Inspiratory Pressure

PO2 and PCO2: Partial Pressure of Oxygen and Carbon Dioxide, respectively

PRVC/PCVG Mode: Pressure Regulated Volume Control/Pressure Controlled Volume Guaranteed Mode

PS/PS Mode: Pressure Support/Pressure Support ventilation mode

RN: Registered Nurse

RR: Respiratory Rate

TV: Tidal Volume

As shown, each protocol goes through the various rules by posing Boolean (yes-no, shown as “Y” and “N”) questions and then setting treatment levels or recommending procedures. In practice, some of these questions may be self-answered by the program software and some questions may be posed to and answered by a caregiver. Additionally as shown, the procedures may include changing the value of variables either based on the results of the Boolean questions or based on the value of other variables (e.g., values drawn from lab tests, vital signs, etc.). Some end procedures in the example algorithms, labeled 502 and 504 in FIG. 5B, 602 and 604 in FIG. 6D, 702 in FIG. 7A, 704 and 706 in FIG. 7B, 902 in FIG. 9A, and 904 in FIG. 9C are shown. In some cases, the end procedures involve the procurement of donated organs. Such steps invariably require a surgeon to be contacted. Other treatment procedures may be accomplished by other staff unless specified. The arrangement and design of the elements of the systems and methods as shown in the exemplary embodiments are illustrative only. Although only a few embodiments of the present disclosure have been described in detail, those skilled in the art who review this disclosure will readily appreciate that many modifications are possible without materially departing from the novel teachings and advantages of the subject matter recited.

In addition to the example procedures shown in FIGS. 5A-18, the following procedures are given as examples of protocols that may be used in example embodiments. As with the examples shown in the figures, numerous changes may be made to the following algorithms without departing from the novelty of the techniques and associated systems that could embody or be used in combination with the following algorithms.

Vascular Volume Maintenance and Correction

The maintenance of vascular volume is performed using the 4-2-1 rule based on weight in kg. The patient weight in kg is obtained.

If the weight is >20 kg, 20 is subtracted from the patient weight. The resultant value is added to 60 to determine the rate of maintenance IV fluid to be given.

If the weight is >10 kg but less than 20 kg, 10 is subtracted from the patient weight. The resultant value is multiplied by 2 and added to 40 to determine the rate of maintenance IV fluid to be given.

If the weight is <10 kg, the value is multiplied by 4 to determine the rate of maintenance IV fluid to be given.

Generally, 0.9% normal saline+20 meq potassium or lactated ringers is the maintenance IV fluid.

Additional volume is given as needed based on the measured or otherwise calculated central venous pressure (CVP). If the CVP is elevated beyond the goal range, a diuretic is given to help correct the extra vascular volume present. A CVP correction is amended to account for elevated intrathoracic pressures from positive pressure ventilation. That CVP correction is described in the formula:

Corrected CVP=measured CVP in mmHg−((0.736*mean airway pressure in cmH2O)*correction coefficient #1). 0.736 is the conversion value from mmHg to cmH2O.

The corrected CVP is then placed into a volume correction algorithm described in the formula:

Volume Correction=(lowest corrected acceptable CVP−corrected CVP)*(correction coefficient #2)*(weight in kg).

The lowest corrected acceptable CVP is a parameter at the low end of the goal range of the corrected CVP. For example, if the goal range of corrected CVP was 7 mmHg to 12 mmHg, the lowest acceptable CVP value would be 7 mmHg. The highest acceptable corrected CVP would be 12 mmHg.

If the CVP is greater than the lowest acceptable measured CVP, no fluid is delivered because Volume Correction is negative number. If the corrected CVP is greater than the highest acceptable corrected CVP medications, devices, or both are used to remove excess fluid.

For simplicity, the Volume Correction calculation is rounded to the nearest 10 ml in adults, to the nearest 1 ml in children older than 1 year, and to the nearest 1/10^{th} of a ml in children less than or equal to 1 year. The volume is given as bolus at a specified infusion rate. The total volume to be infused may be capped per the desired protocol so that the full amount of the volume is not delivered should there be concern about the potential for fluid overload. Once the infusion is complete a new CVP is obtained and the calculation and subsequent fluid adjustments are performed again.

Blood Product Correction

Packed Red Blood Cells (PRBCs) are a blood component that makes up most of the oxygen carrying capacity of the blood. The following algorithm corrects for hematocrit or hemoglobin levels that are too low.

Step 1: Obtain hematocrit or hemoglobin.

Step 2: If hematocrit is greater than the lowest acceptable hematocrit there is no need to transfuse. Similarly, if hemoglobin is greater than the lowest acceptable hemoglobin there is no need to transfuse. Return to Step 1 at the desired recheck frequency.

Step 3: Subtract the hematocrit or hemoglobin from the lowest acceptable hematocrit or hemoglobin respectively. If using hemoglobin skip to Step 5.

Step 4: Divide resultant hematocrit subtraction by 3.

Step 5: Divide the patient weight in kg by 70.

Step 6: Multiply the subtracted hemoglobin value or the value in Step 4 (if hematocrit is used) by the result from step 5.

Step 7: Round up to the nearest integer for patients greater than or equal to a preferred weight in kg. For patients under the preferred weight in kg, the value is not rounded but rather is in ml as a function of the volume of the PRBC unit size. The result from Step 7 is the number of units (or ml) of PRBCs to transfuse. This value can be capped at a certain parameter for units to be transfused. For example, if the calculation indicates the need for 8 units of PRBCs but the maximum units that will be allowed for transfusion is 6 units, the value to be transfused is capped at 6 units. Upon completing the transfusion return to Step 1.

Fresh Frozen Plasma (FFP) is a blood product used to help patients stop bleeding. The International Normalized Ratio (INR) is a lab value that measures the blood's ability to clot. The algorithm corrects for an INR that is too high making patients prone to bleeding.

Step 1: Obtain an INR.

Step 2: If the INR is less than the highest acceptable INR there is no need to transfuse FFP. Return to Step 1 at the desired recheck frequency.

Step 3: Subtract the measured INR from the highest acceptable INR.

Step 4: Obtain the patient's weight in kg and divide by 70.

Step 5: Multiply the resultant values from Steps 3 and 4.

Step 6: Multiply the resultant value from Step 5 by a correction coefficient to obtain the number of units to transfuse. Round up to the nearest integer in units to be transfused for patients greater than or equal to a preferred weight in kg. For patients under the preferred weight in kg, the value is not rounded but rather is delivered in ml as a function of the volume of the FFP unit size. This value can be capped at a certain parameter for units to be transfused. For example, if the calculation indicates the need for 16 units of FFP but the maximum units that will be allowed for transfusion is 12 units, the value to be transfused is capped at 12 units. Upon completing the transfusion return to Step 1.

Platelets are a blood component that helps the blood to clot. The following algorithm corrects for platelet levels that are too low.

Step 1: Obtain a platelet count.

Step 2: If the platelet count is greater than the lowest acceptable platelet count there is no need to transfuse. Return to Step 1 at the desired recheck frequency.

Step 3: Subtract the measured platelet count with the lowest acceptable platelet count.

Step 4: Divide the value in Step 3 by the number of platelet packs per bag. For example, it is common to order a “4-pack” of platelets delivered for transfusion in a single bag.

Step 5: Obtain the patient's weight in kg and divide by 70.

Step 6: Multiply the resultant values from Steps 4 and 5. Round up to the nearest integer in packs to be transfused for patients greater than or equal to a preferred weight in kg. For patients under the preferred weight in kg, the value is not rounded but rather is delivered in ml as a function of the volume of the platelet pack unit size. This value can be capped at a certain parameter for units to be transfused. For example, if the calculation indicates the need for 8 “4-packs” of platelets but the maximum units that will be allowed for transfusion is 5 “4-packs”, the value to be transfused is capped at 5 “4-packs”. Upon completing the transfusion return to Step 1.

Blood Pressure Correction Algorithm (Shown in FIGS. 10-18)

Mean Arterial Pressure (MAP) and Heart Rate (HR) are vital sign parameters that are integrally related to vascular volume, pain, and agitation. The following algorithm corrects patient blood pressure and heart rate related problems. Any drug that the patient has had a drug reaction or allergy to will be excluded as an option from the algorithm. A delay before returning to Step 1 is adjusted according to the pharmacokinetics of the drug being used to prevent rapid changes before the effect of the drug can be realized. Note that this algorithm assumes that fluid/volume status is being controlled simultaneously with the vascular volume, blood product, and free water deficit correction algorithms listed previously algorithm. Note the values of MAP and HR can be replaced by whichever goal values are desired. The values written in this algorithm is for illustrative purposes. Note that the <value may need to be 1 greater than the >value for the same unit of measure. The order in which the drugs are delivered is changeable and some listed here are done so for illustrative purposes. Note that some medications control MAP and HR up, some control MAP and HR down, some control MAP up with little change to HR, some control MAP down with little change to HR, some control HR up with little change to MAP, and some control HR down with little change to MAP. The subroutines use example drugs or procedures to control for these MAP and HR parameters. Furthermore, these algorithms may also include parameters to control for Cardiac Output (CO), Cardiac Index (CI), Systemic Vascular Resistance (SVR), and for other factors such as Stroke Volume Variability (SVV) and Central Venous Pressure (CVP). These parameters can be used either in concert with or independent of HR and MAP to obtain optimal blood flow and cardiac function.

Begin Main Routine

Step 1: If MAP<70, go to Step 6

Step 2: If MAP>90, go to Step 9

Step 3: If HR>110, gosub (Subroutine for high HR)

Step 4: If HR<40, gosub (Subroutine for low HR)

Step 5: Go to Step 1

Step 6: If Decadron has not been given in the past 24 hours and a Serum Cortisol level has not been obtained in the past 24 hours, obtain a stat Serum Cortisol.

Step 7: If HR>100, gosub (Subroutine for low MAP and high HR)

Step 8: If HR<101, gosub (Subroutine for low MAP and low/normal HR)

Step 9: If HR>100, gosub (Subroutine for high MAP and HR)

Step 10: If HR<101, gosub (Subroutine for high MAP and low/normal HR) [End main routine]

Subroutine for Low MAP and High HR

If Serum Cortisol is >20 and <24, give Hydrocortisone 25 mg IV Q8 hours

If Serum Cortisol is >12 and <21, give Hydrocortisone 50 mg IV Q8 hours

If Serum Cortisol is <13, give Hydrocortisone 100 mg IV Q8 hours

If an Isoproterenol infusion is running, decrease Isoproterenol infusion by the given rate and return to Step 1

If a Nicardipine infusion is running, decrease Nicardipine infusion by the given rate and return to Step 1

If a Nitroprusside infusion is running, decrease Nitroprusside infusion by the given rate and return to Step 1

If a Diltiazem infusion is running, decrease Diltiazem infusion by the given rate and return to Step 1

If a Labetalol infusion is running, decrease Labetalol infusion by the given rate and return to Step 1

If a Vasopressin infusion is not running, start Vasopressin at 0.04 units/min and return to Step 1

If a Vasopressin infusion is running <max rate, increase Vasopressin infusion at given value and return to Step 1

If a Norepinephrine infusion is not running, start Norepinephrine infusion at starting value and return to Step 1

If a Norepinephrine infusion is running <max rate, increase Norepinephrine infusion at given value and return to Step 1

If a Phenylephrine infusion is not running, start Phenylephrine infusion at starting value and return to Step 1

If a Phenylephrine infusion is running <max rate, increase Phenylephrine infusion at given value and return to Step 1

If the Levothyroxine protocol is not running, gosub levothyroxine protocol and return to Step 1

If a Dobutamine infusion is not running, start Dobutamine infusion at starting value and return to Step 1

If a Dobutamine infusion is running, <max rate, increase Dobutamine infusion at given value and go to Step 1

If a Dopamine infusion is not running, start Dopamine infusion at starting value and return to Step 1

If a Dopamine infusion is running <max rate, increase Dopamine infusion at given value and return to Step 1

If an Epinephrine infusion is not running, start Epinephrine infusion at starting value and return to Step 1

If an Epinephrine infusion is running <max rate, increase Epinephrine infusion at given value and return to Step 1

Call physician for management of other causes that could be contributory to decreased MAP and return to Step 1 [End subroutine.]

Subroutine for Low MAP and Low/Normal HR

If Serum Cortisol is >20 and <24, give Hydrocortisone 25 mg IV Q8 hours

If Serum Cortisol is >12 and <21, give Hydrocortisone 50 mg IV Q8 hours

If Serum Cortisol is <13, give Hydrocortisone 100 mg IV Q8 hours

If a Diltiazem infusion is running, decrease Diltiazem infusion by the given rate and return to Step 1

If a Labetalol infusion is running, decrease Labetalol infusion by the given rate and return to Step 1

If a Nicardipine infusion is running, decrease Nicardipine infusion by the given rate and return to Step 1

If a Nitroprusside infusion is running, decrease Nitroprusside infusion by the given rate and return to Step 1

If an Isoproterenol infusion is running, decrease Isoproterenol infusion by the given rate and return to Step 1

If a Dopamine infusion is not running, start Dopamine infusion at starting value and return to Step 1

If a Dopamine infusion is running <max rate, increase Dopamine infusion at given value and return to Step 1

If a Dobutamine infusion is not running, start Dobutamine infusion at starting value and return to Step 1

If a Dobutamine infusion is running, <max rate increase Dobutamine infusion at given value and return to Step 1

If the Levothyroxine protocol is not running, gosub levothyroxine protocol and return to Step 1

If an Epinephrine infusion is not running, start Epinephrine infusion at starting value and return to Step 1

If an Epinephrine infusion is running <max rate, increase Epinephrine infusion at given value and return to Step 1

If a Vasopressin infusion is not running, start Vasopressin at 0.04 units/min and return to Step 1

If a Vasopressin infusion is running <max rate, increase Vasopressin infusion at given value and return to Step 1

If a Norepinephrine infusion is not running, start Norepinephrine infusion at starting value and return to Step 1

If a Norepinephrine infusion is running <max rate, increase Norepinephrine infusion at given value and return to Step 1

If a Phenylephrine infusion is not running, start Phenylephrine infusion at starting value and return to Step 1

If a Phenylephrine infusion is running <max rate, increase Phenylephrine infusion at given value and return to Step 1

Call physician for management of other causes that could be contributory to decreased MAP and return to Step 1 [End subroutine.]

Subroutine for High MAP and HR

If the patient is uncomfortable, gosub (Comfort Care Subroutine) and upon return, return to Step 1

If an Epinephrine infusion is running, decrease Epinephrine infusion by given value and return to Step 1

If a Dopamine infusion is running, decrease Dopamine infusion by given value and return to Step 1

If a Dobutamine infusion is running, decrease Dobutamine infusion by given value and return to Step 1

If an Isoproterenol infusion is running, decrease Isoproterenol infusion by the given rate and return to Step 1

If a Phenylephrine infusion is running, decrease Phenylephrine infusion by given value and return to Step 1

If a Norepinephrine infusion is running, decrease Norepinephrine infusion by given value and return to Step 1

If a Vasopressin infusion is running, decrease Vasopressin infusion by given value and return to Step 1

If anti-hypertensive prn medications been given 6 times in 2 hours and HR is >60, gosub (Antihypertensive infusion subroutine for high MAP and HR)

If anti-hypertensive prn medications been given 6 times in 2 hours and HR is <61, gosub (Antihypertensive infusion subroutine for high MAP and low/normal HR)

If HR is >60, give Labetalol 10 mg IV and return to Step 1

IF HR is <61, give Hydralazine 10 mg IV and return to Step 1 [End subroutine.]

Subroutine for High MAP and Low/Normal HR

If the patient is uncomfortable, gosub (Comfort Care Subroutine) and upon return, return to Step 1

If a Phenylephrine infusion is running, decrease Phenylephrine infusion by given value and return to Step 1

If a Norepinephrine infusion is running, decrease Norepinephrine infusion by given value and return to Step 1

If a Vasopressin infusion is running, decrease Vasopressin infusion by given value and return to Step 1

If an Epinephrine infusion is running, decrease Epinephrine infusion by given value and return to Step 1

If a Dopamine infusion is running, decrease Dopamine infusion by given value and return to Step 1

If a Dobutamine infusion is running, decrease Dobutamine infusion by given value and return to Step 1

If an Isoproterenol infusion is running, decrease Isoproterenol infusion by the given rate and return to Step 1

If anti-hypertensive prn medications been given 6 times in 2 hours and HR is >60, gosub (Antihypertensive infusion subroutine for high MAP and HR)

If anti-hypertensive prn medications been given 6 times in 2 hours and HR is <61, gosub (Antihypertensive infusion subroutine for high MAP and low/normal HR)

If HR is >60, give Labetalol 10 mg IV and return to Step 1

IF HR is <61, give Hydralazine 10 mg IV and return to Step 1 [End subroutine.]

Antihypertensive Infusion Subroutine for High MAP and HR

If a labetalol infusion has not been started, start Labetalol at 20 mg/hr and return to Step 1

If a labetalol infusion <max rate, increase Labetalol infusion at given rate and go return Step 1

If a Diltiazem infusion has not been started, start Diltiazem infusion at starting rate and go to Step 1

If a Diltiazem infusion <max rate, increase Diltiazem infusion at given rate and go to Step 1

If the patient does not have (liver or kidney dysfunction) and if a Nitroprusside infusion has not been started, start the Nitroprusside infusion at the starting value and return to Step 1

If the patient does not have (liver or kidney dysfunction) and if a Nitroprusside infusion is running <3 mcg/kg/min, increase Nitroprusside by the given value and return to Step 1

If a Nicardipine infusion has not been started, start the Nicardipine infusion at the starting value and return to Step 1

If a Nicardipine infusion is running <max rate, increase Nicardipine by the given value and return to Step 1

Call physician for management of elevated MAP and return to Step 1 [End subroutine.]

Antihypertensive Infusion Subroutine for High MAP and Low/Normal HR

If the patient does not have (liver or kidney dysfunction) and if a Nitroprusside infusion has not been started, start the Nitroprusside infusion at the starting value and return to Step 1:

If the patient does not have (liver or kidney dysfunction) and if a Nitroprusside infusion is running <3 mcg/kg/min, increase Nitroprusside by the given value and return to Step 1

If a Nicardipine infusion has not been started, start the Nicardipine infusion at the starting value and return to Step 1

If a Nicardipine infusion is running <max rate, increase Nicardipine by the given value and return to Step 1

Call physician for management of elevated MAP and return to Step 1 [End subroutine.]

Subroutine for High HR

If the patient is uncomfortable, gosub (Comfort Care Subroutine) and upon return, return to Step 1

If Decadron has not been given in the past 24 hours and a Serum Cortisol level has not been obtained in the past 24 hours, obtain a stat Serum Cortisol

If Serum Cortisol is >20 and <24, give Hydrocortisone 25 mg IV Q8 hours

If Serum Cortisol is >12 and <21, give Hydrocortisone 50 mg IV Q8 hours

If Serum Cortisol is <13, give Hydrocortisone 100 mg IV Q8 hours

If ABG hasn't been obtained in the past 6 hours, obtain ABG

If ABG shows an A-a gradient that is high, call physician for management of possible pulmonary embolism

If ABG shows evidence of a respiratory acidosis, gosub (Donor Lung Protocol)

If ABG shows evidence of a metabolic acidosis, call physician for management of metabolic acidosis

If anion gap is large and there is evidence of an acidosis, send blood lactate and blood ketones and notify physician that those labs were sent.

If an Isoproterenol infusion is running, decrease Isoproterenol infusion by the given rate and return to Step 1

If an Epinephrine infusion is running, decrease Epinephrine infusion by given value and return to Step 1

If a Dopamine infusion is running, decrease Dopamine infusion by given value and return to Step 1

If a Dobutamine infusion is running, decrease Dobutamine infusion by given value and return to Step 1

If a Norepinephrine infusion is running, decrease Norepinephrine infusion by given value and return to Step 1

Call physician for management of elevated HR and return to Step 1 [End subroutine.]

Subroutine for Low HR

If a Dobutamine infusion is not running, start Dobutamine infusion at starting value and go to Step 1

If a Dobutamine infusion is running <max rate, increase Dobutamine infusion at given value and go to Step 1

If an Isoproterenol infusion is not running, start Isoproterenol infusion at starting value and go to Step 1

If an Isoproterenol infusion is running <max rate, increase Isoproterenol infusion at given value and go to Step 1

If an Epinephrine infusion is not running and MAP<85, start Epinephrine infusion at starting value and go to Step 1

If an Epinephrine infusion is running, <max rate and MAP<85, increase

Epinephrine infusion at given value and go to Step 1

Give Atropine 1 mg and Notify MD of low HR. [End subroutine.]

Comfort Care Algorithm for Pain and Agitation

This algorithm uses a narcotic and a benzodiazepine to control pain and agitation respectively. Note that multiple narcotics are not used together for pain control although the doses and frequencies of 3 narcotics are described here. Sedatives such as midazolam are used in concert with narcotics as needed for agitation management. Note that other orders such as those for excess salivation are included as well. Atropine 1% Ophthalmic Solution can be delivered sublingually every 30 minutes as needed for oral secretion management. A 1.5 mg scopolamine patch can also be used every 72 hours as needed for oral secretions if needed.

For Pain Management Morphine, Hydromorphone, or Fentanyl are described.

Morphine

Step 1: If the patient does not have any narcotic medications currently infusing, bolus 0.1 mg/kg capped at a maximum dose if pain is identified verbally or nonverbally.

Step 2: Initiate the infusion at 0.1 mg/kg/hr. capped at a maximum infusion rate if pain is identified verbally or nonverbally.

Step 3: Increase infusion rate by 20% every 4 hours if the patient describes pain or displays non-verbal signs of pain as rated by a scale such as the CNPI.

Step 4: Bolus Morphine at 50% of current infusion rate every 30 minutes if the patient describes pain or displays non-verbal signs of pain as rated by a scale such as the CNPI.

Hydromorphone

Step 1: If the patient does not have any narcotic medications currently infusing, bolus 0.05 mg/kg capped at a maximum dose if pain is identified verbally or nonverbally.

Step 2: Initiate the infusion at 0.015 mg/kg/hr. capped at a maximum infusion rate if pain is identified verbally or nonverbally.

Step 3: Increase infusion rate by 20% every 4 hours if the patient describes pain or displays non-verbal signs of pain as rated by a scale such as the CNPI.

Step 4: Bolus Hydromorphone at 50% of current infusion rate every 60 minutes if the patient describes pain or displays non-verbal signs of pain as rated by a scale such as the CNPI.

Fentanyl

Step 1: If the patient does not have any narcotic medications currently infusing, bolus 2 mcg/kg capped at a maximum dose if pain is identified verbally or nonverbally.

Step 2: Initiate the infusion at 0.5 mcg/kg/hr. capped at a maximum infusion rate if pain is identified verbally or nonverbally.

Step 3: Increase infusion rate by 20% every 4 hours if the patient describes pain or displays non-verbal signs of pain as rated by a scale such as the CNPI.

Step 4: Bolus Fentanyl at 50% of current infusion rate every 20 minutes if the patient describes pain or displays non-verbal signs of pain as rated by a scale such as the CNPI.

Midazolam

Step 1: If the patient does not have any sedative medications currently infusing, bolus 0.1 mg/kg capped at a maximum dose if restlessness is identified.

Step 2: Initiate the infusion at 0.05 mg/kg/hr. capped at a maximum infusion rate if restlessness is identified.

Step 3: Increase infusion rate by 20% every 4 hours if the patient describes pain or displays non-verbal signs of pain as rated by a scale such as the CNPI.

Step 4: Bolus midazolam at current infusion rate every 120 minutes if the patient shows evidence of restlessness.

In the subject description, the word “exemplary” is used to mean serving as an example, instance or illustration. Any embodiment or design described herein as “exemplary” is not necessarily to be construed as preferred or advantageous over other embodiments or designs. Rather, use of the word exemplary is intended to present concepts in a concrete manner. Accordingly, all such modifications are intended to be included within the scope of the present disclosure. The order or sequence of any process or method steps may be varied or re-sequenced according to alternative embodiments. Any means-plus-function clause is intended to cover the structures described herein as performing the recited function and not only structural equivalents but also equivalent structures. Other substitutions, modifications, changes, and omissions may be made in the design, operating conditions, and arrangement of the preferred and other exemplary embodiments without departing from scope of the present disclosure or from the scope of the appended claims.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

1. A non-transitory computer-readable medium having stored thereon instructions executable by a processing system to cause the processing system to perform functions, the functions comprising:
receiving heath data representing patient vital statistics;
using preset treatment protocols to generate a recommended treatment action based on the received health data, wherein the preset treatment protocols identify a medical caregiver associated with the recommended treatment action; and
in response to generating the recommended treatment action, automatically causing a notification system to contact the identified medical caregiver.

2. The computer-readable medium of claim 1, wherein preset treatment protocols comprise patient-specific protocols, the functions further comprising:
receiving indications of the patient-specific rules from a user-interface; and
responsively setting the patient-specific protocols in the preset treatment protocols.

3. The computer-readable medium of claim 2, wherein the received indications of the patient-specific needs comprise a selection of a patient profile from a set of preprogrammed patient profiles.

4. The computer-readable medium of claim 1, wherein preset treatment protocols comprise patient-specific protocols, the functions further comprising:
receiving indications of physician-specific rules from a user-interface; and
responsively setting the patient-specific protocols in accordance with the physician-specific rules in the preset treatment protocols.

5. The computer-readable medium of claim 1, wherein the health data is received directly from one or more medical monitoring systems.

6. The computer-readable medium of claim 4, wherein the functions further comprise periodically requesting updated health data from the one or more medical monitoring systems.

7. The computer-readable medium of claim 1, wherein the patient vital statistics comprise readings of blood pressure, blood transfusions, free water deficit, volume status, blood sugar, and ventilation.

8. The computer-readable medium of claim 1, wherein the preset treatment protocols identify different medical caregivers associated with different recommended treatment actions.

9. The computer-readable medium of claim 1, wherein the identified medical caregiver is a physician, and wherein the recommended treatment action comprises only contacting the physician.

10. The computer-readable medium of claim 1, wherein the identified medical caregiver is a nursing professional, the functions further comprising facilitating presentation of the recommended treatment action to the nursing professional.

11. The computer-readable medium of claim 1, wherein the notification system comprises an intercom system, and wherein the notification system contacts the medical caregiver by causing the intercom system to play an announcement associated with the medical caregiver.

12. The computer-readable medium of claim 1, wherein the notification system comprises an external communication network, and wherein the notification system contacts the medical caregiver by causing an alert message to be transmitted to an address on the communication network associated with the medical caregiver.

13. A method comprising:
receiving, at a processing system, heath data representing patient vital statistics;
the processing system using preset treatment protocols to generate a recommended treatment action based on the received health data, wherein the preset treatment protocols identify a medical caregiver associated with the recommended treatment action; and
in response to generating the recommended treatment action, the processing system automatically causing a notification system to contact the identified medical caregiver.

14. The method of claim 13, wherein preset treatment protocols comprise patient-specific protocols, the method further comprising:
receiving indications of the patient-specific rules from a user-interface; and
responsively setting the patient-specific protocols in the preset treatment protocols.

15. The method of claim 14, wherein the received indications of the patient-specific needs comprise a selection of a patient profile from a set of preprogrammed patient profiles.

16. The method of claim 13, wherein the notification system comprises an intercom system, and wherein the notification system contacts the medical caregiver by causing the intercom system to play an announcement associated with the medical caregiver.

17. A processing system comprising:
a computer-readable medium;
a communication interface; and
a processor configured to:
receive heath data representing patient vital statistics;
use preset treatment protocols stored in the computer-readable medium to generate a recommended treatment action based on the received health data, wherein the preset treatment protocols identify a medical caregiver associated with the recommended treatment action; and
in response to generating the recommended treatment action, automatically cause, via the communication interface, a notification system to contact the identified medical caregiver.

18. The processing system of claim 17, wherein preset treatment protocols comprise patient-specific protocols, and wherein the processor is further configured to:
receive indications of the patient-specific rules from a user-interface; and
responsively set the patient-specific protocols in the preset treatment protocols.

19. The processing system of claim 17, wherein
the received indications of the patient-specific needs comprise a selection of a patient profile from a set of preprogrammed patient profiles.

20. The processing system of claim 17, wherein
the notification system comprises an intercom system, and wherein the notification system contacts the medical caregiver by causing the intercom system to play an announcement associated with the medical caregiver.
